(19) United States
(12) Patent Application Publication
(10) Pub. No.: US 2011/0017599 A1
(43) Pub. Date: Jan. 27, 2011

(54) TARGET RECOGNITION MOLECULE AND A METHOD FOR IMMOBILIZING THE SAME

(75) Inventor: Yuichiro SHIMIZU, Osaka (JP)

Correspondence Address: NIXON & VANDERHYE, PC, 901 NORTH GLEBE ROAD, 11TH FLOOR, ARLINGTON, VA, 22203, US

(73) Assignee: Sharp Kabushiki Kaisha, Osaka (JP)

(21) Appl. No.: 12/842,803

(22) Filed: Jul. 23, 2010

Related U.S. Application Data
(63) Continuation of application No. 12/778,418, filed on May 12, 2010, now abandoned.

(30) Foreign Application Priority Data
May 14, 2009 (JP) 2009-117873

(51) Int. Cl. G01N 27/447; C07K 2/00

(52) U.S. Cl. 204/548; 530/300; 530/322; 204/600

(57) ABSTRACT

A novel target recognition molecule is provided which has a electrostatic property whereby such target recognition molecules can be densely brought together in a self-assembly manner in a predetermined region of an analytical chip and, in addition, can be reversibly or irreversibly stably immobilized there. This target recognition molecule has a target recognition peptide segment as a specific binding site for a target substance, an electrostatically-charged segment which is provided with three or more electrostatically-charged functional groups capable of being electrically charged with charges of the same polarity in the same solution and which has no functional groups that become electrically charged to different polarities in the same solution, and a connecting segment which chemically links with the target recognition peptide segment and with the electrostatically-charged segment for establishing a connection between both the segments.

This application is a continuation of U.S. patent application Ser. No. 12/778,418 filed May 12, 2010 which claims priority to Japanese Application No. 2009-117873, filed 14 May 2009, the entire contents of which is hereby incorporated by reference.

### BACKGROUND OF THE INVENTION

1. Field of the Invention

This invention generally relates to a target recognition molecule having a binding site which specifically interacts with a target substance, and it more specifically relates to a target recognition molecule which is imparted with an electrostatic property so that it is densely self-assembled and immobilized at a specific site of an analytical chip.

2. Description of the Prior Art

In recent years, an analytical chip formed by immobilization of target recognition molecules within a chip has been used for the analysis of a biologic substance such as protein.

As a target recognition molecule for use in such an analytical chip, there has been used a substance which specifically selectively interacts with a target substance, and naturally-derived antibodies have been used in the past. However, recently, artificial antibodies formed of synthetic peptides or other like compounds have been used from the aspect of their long-term storability, productivity and so on.

Such a type of analytical chip provided with a substance specifically selectively interactive with a target substance is easy to manipulate and requires no high level of analytical skill. In addition, there is an advantage capable of assaying a target substance in a short period of time with a less test substance volume. On the other hand, for example, it is not easy to properly immobilize and hold a required amount of target recognition molecule at a predetermined spot, therefore there is not always obtained an assay accuracy, reliability, or reproducibility of satisfactory level.

In regard to the method for immobilization of a target recognition molecule, a variety of methods have been proposed heretofore. For example, one known method is to cause a target recognition molecule to be physically adsorbed on the surface of a base material, and another known method is to cause a target recognition molecule to be covalently linked to the surface of a base material. In addition, there is still another known method in which a target recognition molecule is immobilized on the surface of a minute bead and the minute bead is placed in a microchannel. Furthermore, there are other known methods as set forth in the following conventional art literatures.

### Citation List

### Patent Literature

Patent Literature 1: JP-T-2007-504471
Patent Literature 2: JP-A-2003-344396
Patent Literature 3: JP-A-2006-266831
Patent Literature 4: JP-T-04-501605
Patent Literature 5: JP-A-2000-266716
Patent Literature 6: JP-A-2006-71324

### SUMMARY OF THE INVENTION

For the case of an analytical chip which is formed such that it is provided with a substance specifically selectively interactive with a target substance, its analytical performance depends much on whether the immobilization density or the immobilization state of target recognition molecules is good or bad and, in addition, the productivity of a analytical chip depends much on the efficiency of immobilization. Accordingly, an object of the present invention is to provide a novel target recognition molecule in that the target recognition molecule itself is imparted with a function of high density immobilization. In addition, another object of the present invention is to provide a technology for efficiently immobilizing a target recognition molecule onto a base material.

If the immobilization efficiency of target recognition molecules is low in the fabrication of an analytical chip, this requires more use of target recognition molecules as a source material. The waste of target recognition molecules results in a cost increase, and the immobilization of low efficiency contributes to a productivity decrease. Besides, if the immobilization density is low, this will not provide high analytical sensitivity. In addition, if there are differences in immobilization density between individual analytical chips, this results in considerable reliability degradation. Therefore, there have been demands for means by which target recognition molecules can be rapidly, densely and with high reproducibility immobilized at a predetermined site within an analytical chip.

In addition, if the target recognition molecule is one that is poor in chemical/physical stability, this gives rise to a problem that an analytical chip, formed by immobilization of such a substance, soon becomes unserviceable. One of methods for overcoming this problem is a method in which target recognition molecules are in situ immobilized at the time of analysis. To this end, however, there is required a means capable of simply immobilizing target recognition molecules on the spot of analysis.

The inventor of the present invention conducted intensive researches with view to overcoming these problems and finally accomplished the present invention which makes it possible for a target recognition molecule itself to become a molecule capable of high density immobilization.

The present invention is directed to a novel target recognition molecule into which an electrostatically-charged segment has been incorporated, and a group of aspects of the present invention are configured as described below.

(1) In accordance with a first aspect of the invention, there is provided a target recognition molecule including: a target recognition peptide segment as a specific binding site for a target substance; an electrostatically-charged segment which is provided with electrostatically-charged functional groups capable of being electrically charged with charges of the same polarity in the same solution; and a connecting segment which chemically links with the target recognition peptide segment and with the electrostatically-charged segment for establishing a connection between both the segments.

(2) In accordance with a second aspect of the present invention, there is provided a target recognition molecule according to the aforesaid first aspect wherein the electrostatically-charged segment comprises three or more electrostatically-charged functional groups that become electrically charged with charges of the same polarity in the same solution.

(3) In accordance with a third aspect of the present invention, there is provided a target recognition molecule according to either the aforesaid first or second aspect wherein the isoelectric point of the target recognition peptide segment is 6 or less, and wherein the electrostatically-charged functional groups of the electrostatically-charged segment are functional groups which become negatively electrically charged in an aqueous solution of a pH value of 7 or greater.

(4) In accordance with a fourth aspect of the present invention, there is provided a target recognition molecule according to either the aforesaid first or second aspect wherein the isoelectric point of the target recognition peptide segment is 8 or greater, and wherein the electrostatically-charged functional groups of the electrostatically-charged segment are functional groups which become positively electrically charged in an aqueous solution of a pH value of 7 or less.

Here, by “comprising three or more electrostatically-charged functional groups that become electrically charged with charges of the same polarity” is meant that there may be provided either three or more identical functional groups or three or more functional groups of different types. In addition, what is meant by “the isoelectric point of the target recognition peptide segment” is an isoelectric point (average isoelectric point) which is defined by a value (average value) found as a result of division of a sum value (which is a combined value of the isoelectric points of amino acids corresponding to individual amino acid residues forming a target recognition peptide segment) by the number of the amino acid residues. The isoelectric point of each of the amino acids is as shown in Table 1.

TABLE 1

| amino acid class | abbrev. form | isoelectric point |
|---|---|---|
| alanine | A | 6.00 |
| arginine | R | 10.76 |
| asparagine | N | 5.41 |
| aspartic acid | D | 2.77 |
| cysteine | C | 5.05 |
| glutamine | Q | 5.65 |
| glutamic acid | E | 3.22 |
| glycine | G | 5.97 |
| histidine | H | 7.59 |
| isoleucine | I | 6.05 |
| leucine | L | 5.98 |
| lysine | K | 9.75 |
| methionine | M | 5.74 |
| phenylalanine | F | 5.48 |
| proline | P | 6.30 |
| serine | S | 5.68 |
| threonine | T | 6.16 |
| tryptophan | W | 5.89 |
| tyrosine | Y | 5.66 |
| valine | V | 5.96 |

(5) In accordance with a fifth aspect of the invention, there is provided a target recognition molecule according to any one of the aforesaid first to fourth aspects wherein the electrostatically-charged segment comprises three or more electrostatically-charged functional groups that become electrically charged with charges of the same polarity in the same solution and, in addition, has no functional groups that become electrically charged to different polarities.

Now, a description will be given of how the target recognition molecule according to the present invention is used. Through the description, the technical point of the configuration of a group of aspects of the present invention will be demonstrated.

The target recognition molecule of the present invention has an enhanced property of assembling in the electric field, thereby making it possible to cause, by making use of such a property, target recognition molecules to assemble and be retained in a desired immobilization site. For example, in an analytical chip with a microchannel formed therein, an electrode is formed at a desired location for the assembling and retaining of target recognition molecules. This is followed by application of a voltage to the electrode so that it is given positive or negative electric charges. Under this state, a target recognition molecule containing solution in which target recognition molecules are dissolved is flowed through the channel, whereby the target recognition molecules can be efficiently gathered together on the electrode surface as an immobilization site and, in addition, can be reversibly or irreversibly immobilized there. In the following, a description will be given of this point.

The target recognition molecule according to the fifth aspect of the present invention is provided with three or more electrostatically-charged functional groups that become electrically charged with charges of the same polarity in the same solution and, in addition, has no functional groups that become electrically charged to different polarities. Therefore, if such a target recognition molecule is dissolved in solution, the electrostatically-charged segment becomes electrically charged with charges of the same polarity. Accordingly, when a solution containing target recognition molecules of the present invention is flowed on the charge-applied immobilization site (electrode), target recognition molecules are attracted onto the electrode surface by electrostatic interaction and densely trapped there. This densely assembling state will be held as long as the electrode is electrically charged with charges. Stated another way, the target recognition molecules are reversibly immobilized on the immobilization site.

The above will be described in detail. If a target recognition molecule, provided with a target recognition peptide segment whose isoelectric point is 6 or less and an electrostatically-charged segment whose electrostatically-charged functional groups are those that become negatively electrically charged, is dissolved in a carrier solution having, for example, a pH value of 8, then both the target recognition peptide segment and the electrostatically-charged segment become negatively electrically charged. Here, the requirement of both the third and fifth aspects of the present invention is met by an electrostatically-charged segment as follows. That is, the isoelectric point is 6 or less; the electrostatically-charged functional groups are those that become negatively electrically charged in an aqueous solution having a pH value of 7 or greater; and there are provided three or more electrostatically-charged functional groups that become electrically charged with charges of the same polarity in the same solution and there are no functional groups that become electrically charged to different polarities, whereby the negative charge density of the electrostatically-charged segment is satisfactorily high. Therefore, upon application of positive electric charges to the electrode, the electrostatically-charged segment part is attracted to the electrode and trapped onto the electrode surface.

On the other hand, the requirement of both the fourth and fifth aspects of the present invention is met by an electrostatically-charged segment as follows. That is, the isoelectric point of the target recognition peptide segment is 8 or greater and the electrostatically-charged segment is provided with functional groups that become positively electrically charged and, in addition, has no functional groups that become electrically charged to different polarities. Therefore, if this target recognition molecule is dissolved in a carrier solution of, for example, a pH value of 6, then both the target recognition peptide segment and the electrostatically-charged segment become positively electrically charged and the charge density thereof is satisfactorily high. Therefore, upon application of negative electric charges to the electrode, the electrostatically-charged segment part is attracted to the electrode and trapped onto the electrode surface.

In the target recognition molecule as configured above, either the connecting segment or the electrostatically-charged segment or both function as a spacer for maintaining the distance between the immobilization site (electrode surface) and the target recognition peptide segment as well as as an arm for securing the degree of freedom of the target recognition peptide segment. Therefore, even in a state in which one end of the molecule is immobilized to the electrode, the molecule satisfactorily exhibits its specific recognition ability against a target substance.

(6) In accordance with a sixth aspect of the present invention, there is provided a target recognition molecule according to the aforesaid fifth aspect wherein the target recognition peptide segment contains a cysteine residue and the connecting segment is chemically linked to elemental sulfur of the cysteine residue.

(7) In addition, in accordance with a seventh aspect of the present invention, there is provided a target recognition molecule according to any one of the aforesaid first to fifth aspects wherein one end of the target recognition peptide segment is a cysteine residue and the connecting segment is chemically linked to elemental sulfur of the cysteine residue.

(8) Still in addition, in accordance with an eighth aspect of the present invention, there is provided a target recognition molecule according to any one of the aforesaid first to sixth aspects wherein the connecting segment is chemically linked to a terminal of an amino acid residue of the target recognition peptide segment and the electrostatically-charged segment is chemically linked to a different site of the connecting segment from the aforementioned chemical linkage site.

This configuration impedes the connecting and electrostatically-charged segments from inhibiting the specific property of the target recognition peptide segment to a target substance. That is, the target recognition peptide segment is allowed to easily exhibit a function of selecting a target substance.

(9) Still in addition, in accordance with a ninth aspect of the present invention, there is provided a target recognition molecule according to any one of the aforesaid first to eighth aspects wherein the electrostatically-charged segment is composed of a peptide having three or more amino acid residues.

(10) Still in addition, in accordance with a tenth aspect of the present invention, there is provided a target recognition molecule according to the aforesaid ninth aspect wherein the peptide as the electrostatically-charged segment contains three or more basic amino acid residues, one or more of which are selected from a group composed of arginine and lysine, and contains neither an aspartic acid residue nor a glutamic acid residue.

The electrostatically-charged segment according to this configuration is formed containing three or more particular basic amino acid residues of high isoelectric point, and contains no acidic amino acid residues of low isoelectric point so that it becomes strongly positively charged in a solution ranging from mild alkali to acidic. Therefore, the target recognition molecule having this configuration is suitable for the analysis of a target substance which employs a solution of from mild alkali to acidic.

(11) Still in addition, in accordance with an eleventh aspect of the present invention, there is provided a target recognition molecule according to the aforesaid ninth aspect wherein the peptide as the electrostatically-charged segment contains three or more acidic amino acid residues, one or more of which are selected from a group composed of aspartic acid and glutamic acid, and contains neither an arginine residue nor a lysine residue.

The electrostatically-charged segment according to this configuration is formed containing three or more particular acidic amino acid residues of low isoelectric point, and contains no basic amino acid residues of high isoelectric point so that it becomes strongly negatively charged in a solution from mild acidic to alkali. Therefore, the target recognition molecule having this configuration is suitable for the analysis of a target substance that employs a solution of from mild acidic to alkali.

(12) Still in addition, in accordance with a twelfth aspect of the present invention, there is provided a target recognition molecule according to any one of the aforesaid ninth to eleventh aspects wherein the number of amino acid residues forming the electrostatically-charged segment is larger than the number of amino acid residues forming the target recognition peptide segment.

The target recognition peptide segment is usually composed of a peptide which is a mixture of acidic amino acid, basic amino acid, and neutral amino acid. Therefore, if the electrostatically-charged segment is greater in the number of amino acid residues than the target recognition peptide segment, then the electrostatically-charged segment formed of a unevenly distributed amino acid sequence becomes more strongly electrically charged than the target recognition peptide segment, thereby accomplishing its role.

(13) Still in addition, in accordance with a thirteenth aspect of the present invention, there is provided a target recognition molecule according to any one of the aforesaid first to eighth aspects wherein the electrostatically-charged segment is a segment which has a polyacrylic acid building block represented by the following chemical formula (1) with n being not less than 3 nor more than 150.

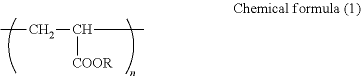

wherein R is H, Na, or K.

(14) Still in addition, in accordance with a fourteenth aspect of the present invention, there is provided a target recognition molecule according to any one of the aforesaid first to eighth aspects wherein the electrostatically-charged segment is a segment having a polystyrene sulfonic acid building block represented by the following chemical formula (2) with n being not less than 3 nor more than 150.

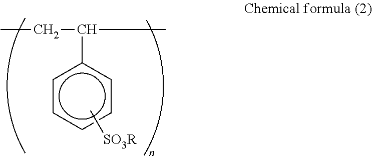

wherein R is H, Na, or K.

(15) Still in addition, in accordance with a fifteenth aspect of the present invention, there is provided a target recognition molecule according to any one of the aforesaid first to eighth aspects wherein the electrostatically-charged segment is a segment having a polyethylenimine building block represented by the following chemical formula (7).

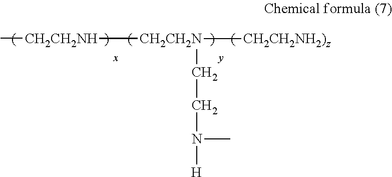

wherein x:y:x=0.5:0.25:0.25 and [x+y+z] is an integer not less than 3 nor more than 150.

(16) Still in addition, in accordance with a sixteenth aspect of the present invention, there is provided a target recognition molecule according to any one of the aforesaid first to eighth aspects wherein the electrostatically-charged segment is a segment having a polyallylamine hydrochloride building block represented by the following chemical formula (8) with n being not less than 3 nor more than 150.

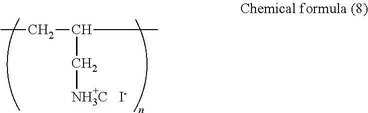

(17) Still in addition, in accordance with a seventeenth aspect of the present invention, there is provided a target recognition molecule according to any one of the aforesaid first to seventh aspects wherein the electrostatically-charged segment is a segment having a polydiallyldimethylammonium chloride building block represented by the following chemical formula (9) with n being not less than 3 nor more than 150.

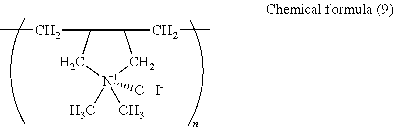

(18) Still in addition, in accordance with an eighteenth aspect of the present invention, there is provided a target recognition molecule according to any one of the aforesaid first to eighth aspects wherein the electrostatically-charged segment is a segment having a polyvinylpyridine building block represented by the following chemical formula (10) with n being not less than 3 nor more than 150.

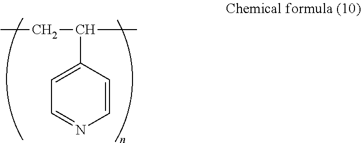

(19) Still in addition, in accordance with a nineteenth aspect of the present invention, there is provided a target recognition molecule according to any one of the aforesaid first to eighth aspects wherein the electrostatically-charged segment is a segment having a polyvinyl sulfate building block represented by the following chemical formula (3) with n being not less than 3 nor more than 150.

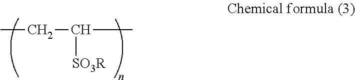

wherein R is H, Na, or K.

(20) Still in addition, in accordance with a twentieth aspect of the present invention, there is provided a target recognition molecule according to any one of the aforesaid first to eighth aspect wherein the electrostatically-charged segment is a segment having a dextran sulfate building block represented by the following chemical formula (4) with n being not less than 1 nor more than 150.

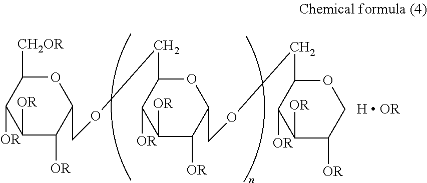

wherein R is SO₃Na or H.

(21) Still in addition, in accordance with a twenty-first aspect of the present invention, there is provided a target recognition molecule according to any one of the aforesaid first to eighth aspects wherein the electrostatically-charged segment is a segment having a chondroitin sulfate building block represented by the following chemical formula (5) with n being not less than 1 nor more than 150.

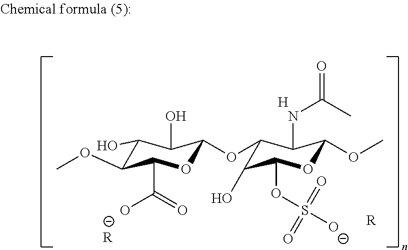

wherein R is H, Na, or K.

(22) Still in addition, in accordance with a twenty-second aspect of the present invention, there is provided a target recognition molecule according to any one of the aforesaid first to eighth aspects wherein the electrostatically-charged segment is a segment having a nucleotide building block represented by the following chemical formula (6) with n being not less than 3 nor more than 150.

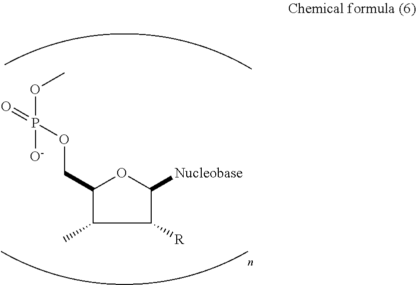

wherein R is H or OH.

Since a nucleotide, composed of a phosphoric acid, a sugar (either ribose (R═OH) or deoxyribose (R═H)), and bases (adenine, cytosine, guanine, thymine (only for deoxyribose), uracil (only for ribose)), has a phosphoric acid content, it becomes electrically charged with negative charges in a basic solution. Therefore, the target recognition molecule of this configuration is suitable for the analysis of a target substance that employs a solution of from alkali to mild acidic. In addition, a single stranded polynucleotide (such as ssDNA and ssRNA) may be used as an electrostatically-charged segment. Alternatively, a double stranded polynucleotide (dsDNA) may be used as an electrostatically-charged segment.

By proper selection of n, the connecting segments, respectively, having the building blocks of the chemical formulas (1)-(10) make it possible that each target recognition peptide segment sufficiently exerts a specific sensibility.

(23) Still in addition, in accordance with a twenty-third aspect of the present invention, there is provided a target recognition molecule according to any one of the aforesaid first to twenty-second aspects wherein the connecting segment has no functional groups that become electrically charged in the solution.

This configuration facilitates molecular design because there is caused no effect on the electrostatic property of an electrostatically-charged segment.

(24) Still in addition, in accordance with a twenty-fourth aspect of the present invention, there is provided a target recognition molecule according to any one of the aforesaid first to twenty-third aspects wherein the target recognition peptide segment comprises a peptide which has not less than 3 nor more than 19 amino acid residues.

For the case of a peptide whose number of amino acids is 2 or less, it is hard for such a peptide to provide a specific recognition capability. However, if the number of amino acids is three or more, it becomes possible to form a peptide capable of exerting a specific target recognition capability. Furthermore, if the number of amino acids is not less than 3 nor more than 19, this facilities the synthesis of a peptide, and for the case of a peptide with a bond number within such a range, its handleability as a target recognition molecule is good.

(25) Still in addition, in accordance with a twenty-fifth aspect of the present invention, there is provided a target recognition molecule according to any one of the aforesaid first to twenty-fourth aspects wherein further chemically linked to the electrostatically-charged segment is a base material immobilizing segment which is provided with a functional group for linkage to a base material.

The target recognition molecule of this configuration can be used as follows. The target recognition molecule, in which a base material immobilizing segment is further connected to the electrostatically-charged segment, has a property of being trapped in an electric field such as an applied electrode surface, and if a solution containing such target recognition molecules is injected into a microchannel in which an electrode is disposed in an immobilization site, the target recognition molecules are densely brought together on the electrode surface. In this state, the functional group (called the “base material immobilizing group”) for establishing a connection to the base material is either in contact with the electrode surface or located in the vicinity of the electrode surface, and brought into contact with the electrode surface by fluctuation of the base material immobilizing segment. Therefore, it becomes possible that the functional group is readily linked to the immobilization site (electrode surface).

To sum up, the use of a target recognition molecule as configured above makes it possible to provide high-density immobilization without fail and once linked, the target recognition molecule will remain held in the immobilization site even if the application of voltage is stopped. This makes it possible to achieve an analytical chip of excellent analytical precision and reliability.

(26) Still in addition, in accordance with a twenty-sixth aspect of the present invention, there is provided a target recognition molecule according to the aforesaid twenty-fifth aspect which is characterized in that the connecting segment is chemically linked, at a linkable site of one end thereof, to the target recognition peptide segment, and is chemically linked, at a linkable site of the other end furthest away from the one end's linkable site, to the electrostatically-charged segment, and that the electrostatically-charged segment is chemically linked, at a linkable site thereof furthest away from the site of its chemical linkage to the connecting segment, to the base material immobilizing segment.

Because of this configuration, the distance between the immobilization site and the target recognition peptide segment increases and the degree of freedom of the target recognition peptide segment increases, whereby the target recognition function will not be easily impeded.

### Invention of Process

There are provided a twenty-seventh and a twenty-eighth aspect of the present invention which relate to immobilization methods for utilizing the target recognition molecules according to the aforesaid first to twenty-fourth aspects while there are provided a twenty-ninth and a thirtieth aspect of the present invention which relate to immobilization methods for utilizing the target recognition molecules according to the aforesaid twenty-fifth and twenty-sixth aspects.

(27) In accordance with a twenty-seventh aspect of the present invention, there is provided a method for immobilizing a target recognition molecule formed in accordance with any one of the aforesaid aspects 3, 5-9, 11, 14, and 19-26 onto an analytical chip provided with an electrode formed in a microchannel, wherein the method comprises: a step in which the target recognition molecule, having a target recognition peptide segment whose isoelectric point is 6 or less and an electrostatically-charged segment whose electrostatically-charged functional groups are those capable of becoming negatively electrically charged, is dissolved in a solution to thereby prepare a target recognition molecule containing solution with an adjusted solution pH value of 7 or greater, and a step in which, with positive electric charges impressed to the electrode in the microchannel, the target recognition molecule containing solution is flowed in the microchannel so that the target recognition molecule is electrically trapped and retained on the surface of the electrode.

(28) In addition, in accordance with a twenty-eighth aspect of the present invention, there is provided a method for immobilizing a target recognition molecule formed in accordance with any one of the aforesaid aspects 4-10, 15-18, and 23-26 onto an analytical chip provided with an electrode formed in a microchannel, wherein the method comprises: a step in which the target recognition molecule, having a target recognition peptide segment whose isoelectric point is 8 or greater and an electrostatically-charged segment whose electrostatically-charged functional groups are those capable of becoming positively electrically charged, is dissolved in a solution to thereby prepare a target recognition molecule containing solution with an adjusted solution pH value of 7 or less, and a step in which, with negative electric charges impressed to the electrode in the microchannel, the target recognition molecule containing solution is flowed in the microchannel so that the target recognition molecule is electrically trapped and retained on the surface of the electrode.

(29) In accordance with a twenty-ninth aspect of the present invention, there is provided a method for immobilizing a target recognition molecule formed in accordance with either the aforesaid twenty-fifth or sixth aspect onto an analytical chip provided with an electrode formed in a microchannel, wherein the method comprises: a step in which the target recognition molecule, having a target recognition peptide segment whose isoelectric point is 6 or less and an electrostatically-charged segment whose electrostatically-charged functional groups are those capable of becoming negatively electrically charged, is dissolved in a solution to thereby prepare a target recognition molecule containing solution with an adjusted solution pH value of 7 or greater, and a step in which, with positive electric charges impressed to the electrode in the microchannel, the target recognition molecule containing solution is flowed in the microchannel so that the target recognition molecule is electrically trapped on the surface of the electrode, thereby chemically linking a base material immobilizing segment of the target recognition molecule with the electrode.

(30) In accordance with a thirtieth aspect of the present invention, there is provided a method for immobilizing a target recognition molecule formed in accordance with either the aforesaid twenty-fifth or sixth aspect onto an analytical chip provided with an electrode formed in a microchannel, wherein the method comprises: a step in which the target recognition molecule, having a target recognition peptide segment whose isoelectric point is 8 or greater and an electrostatically-charged segment whose electrostatically-charged functional groups are those capable of becoming positively electrically charged, is dissolved in a solution to thereby prepare a target recognition molecule containing solution with an adjusted solution pH value of 7 or less, and a step in which, with negative electric charges impressed to the electrode in the microchannel, the target recognition molecule containing solution is flowed in the microchannel so that the target recognition molecule is electrically trapped on the surface of the electrode, thereby chemically linking a base material immobilizing segment of the target recognition molecule with the electrode.

In order for the electrostatically-charged functional groups of the electrostatically-charged segment to become sufficiently dissociated, the solution pH value is preferably 7.3 or greater in the twenty-fifth and twenty-sixth aspects of the present invention while on the other hand the solution pH value is preferably 6.5 or less in the twenty-fourth and twenty-sixth aspects.

### Electrode Base Plate and Detection Device Having a Target Recognition Molecule

(31) There is provided a thirty-first aspect of the present invention with regard to an electrode base plate formed by immobilization of the aforesaid target recognition molecule. This base plate is provided with the following configuration.

The target recognition molecule immobilization electrode base plate comprises a base plate which is provided with an electrode and a target recognition molecule according to any one of the aspects as set forth in the aforesaid paragraphs (1)-(26) immobilized onto the electrode of the base plate.

(32) There is provided a thirty-second aspect of the present invention with regard to a device for detecting a specific molecule which requires the aforesaid target recognition molecule as an essential element. This device is provided with the following configuration.

The specific molecule detection device comprises an electrode, a detector for detecting a target molecule trapped by a target recognition molecule, and a target recognition molecule according to either the twenty-fifth or twenty-sixth aspect as set forth in the aforesaid paragraphs (25)-(26) immobilized onto said electrode.

### Advantageous Effects of the Invention

The target recognition molecule of the present invention is a chemical compound having a structure that it is provided, at one end thereof, with a target recognition peptide segment which specifically interacts with a target substance and at the other end with an electrostatically-charged segment which becomes either positively or negatively electrically charged wherein both the segments are connected together by a connecting segment. In the target recognition molecule of the present invention with this structure, the target recognition peptide segment exhibits a property of specifically recognizing a target substance as a target for analysis while the electrostatically-charged segment exhibits a property of densely assembling onto the applied electrode (immobilization site). Furthermore, the connecting segment and the electrostatically-charged segment prevent the target recognition peptide segment from decreasing in the degree of freedom so that the target recognition peptide segment is allowed to function to sufficiently exert its specific recognition function.

Target recognition molecules according to the present invention, when used, are densely held in an easy and without-fail manner in an immobilization site where the electrode is formed, and such immobilization by electric hold is reversible, thereby achieving significant improvement in the usability of analytical chips. In addition, the dense immobilization of target recognition molecules significantly improves the analytical sensitivity and precision of analytical chips.

Furthermore, in the target recognition molecule of the present invention in which a base material immobilizing segment is linked to the electrostatically-charged segment, the base material immobilizing segment has a functional group that is linked to the base material, thereby making it possible that target recognition molecules are first densely brought together by applying a voltage to a site that requires immobilization and, in this state, the target recognition molecules and the base material are linked together via the base material immobilizing segment. The linkage via the base material immobilizing segment is not released even if the voltage application is stopped, thereby providing a remarkable effect that more ensured dense immobilization can be readily achieved.

### BRIEF DESCRIPTION OF THE DRAWINGS

FIG. 1 is a conceptual diagram showing a connection of constituent elements of a target recognition molecule of the present invention.

FIG. 2 is a conceptual diagram showing a state of how a target recognition molecule shown in FIG. 1 is held by intercharge interaction onto an electrode (immobilization site).

FIG. 3 is a conceptual diagram showing a connection of constituent elements of a target recognition molecule of the present invention which has a base material immobilizing segment.

FIG. 4 is a conceptual diagram showing a state of how a target recognition molecule of the present invention which has a base material immobilizing segment is held by intercharge interaction onto an electrode (immobilization site).

FIG. 5 is a conceptual diagram showing a condition of how a target recognition molecules of the present invention which has a base material immobilizing segment is chemically linked to an electrode as a base material.

FIG. 6 is a diagram showing an example of an analytical chip device which is an application target of a target recognition molecule of the present invention.

### DETAILED DESCRIPTION OF THE INVENTION

Examples for carrying out the present invention will be described successively hereinafter. It is to be understood that the present invention is not limited in application to the following examples and may be carried out with appropriate changes and modifications within the scope of not changing the subject-matter of the present invention.

### First Group of Examples

### Example 1-1

### (Peptide Segment)

As a target recognition peptide segment (abbreviated as “target recognition PS”), there was prepared a protein kinase A (PKA) substrate peptide. This peptide has an amino acid sequence (LRRASLG) and its serine residue is phosphorylated. In addition, the isoelectric point (average value) calculated based on the following Table 1 and mathematical formula (1) is 7.3.

$$\begin{matrix}ISOELECTRIC POINT \\ OF TARGET \\ RECOGNITION PS\end{matrix} = \frac{\begin{matrix}SUM OF ISOELECTRIC POINTS \\ OF AMINO ACIDS \\ CORRESPONDING RESPECTIVELY \\ TO AMINO ACID RESIDUES\end{matrix}}{\begin{matrix}NUMBER OF \\ AMINO ACID RESIDUES\end{matrix} } \qquad Mathematical formula ( 1 )$$

TABLE 1

| amino acid class | abbrev. form | isoelectric point |
|---|---|---|
| alanine | A | 6.00 |
| arginine | R | 10.76 |
| asparagine | N | 5.41 |
| aspartic acid | D | 2.77 |
| cysteine | C | 5.05 |
| glutamine | Q | 5.65 |
| glutamic acid | E | 3.22 |
| glycine | G | 5.97 |
| histidine | H | 7.59 |
| isoleucine | I | 6.05 |
| leucine | L | 5.98 |
| lysine | K | 9.75 |
| methionine | M | 5.74 |
| phenylalanine | F | 5.48 |
| proline | P | 6.30 |
| serine | S | 5.68 |
| threonine | T | 6.16 |
| tryptophan | W | 5.89 |
| tyrosine | Y | 5.66 |
| valine | V | 5.96 |

### (Electrostatically-Charged Segment)

As an electrostatically-charged segment, there was used a peptide (amino acid sequence; DDDDDDDD) comprised of a coupled series of eight aspartic acids which are acidic amino acids. This electrostatically-charged segment has an isoelectric point of 2.77 (average value), and is hydrophilic.

### (Connecting Segment)

As a connecting segment, there was used a [Bis-N-Succinimidyl-(pentaethylene-glycol)ester] (the chemical formula (12)) comprised of five building blocks of polyethylene glycol (n=5) shown in the general chemical formula (11). A succinimide group (NHS) at one end thereof was reached with an amino group of an N-terminal amino acid residue of the aforesaid target recognition peptide segment and a succinimide group at the other end was linked to an amino group of an N-terminal amino acid residue of the aforesaid electrostatically-charged segment.

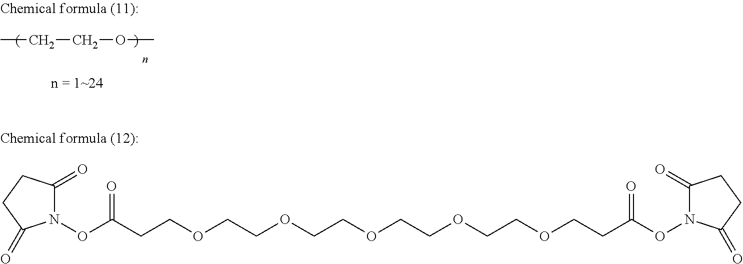

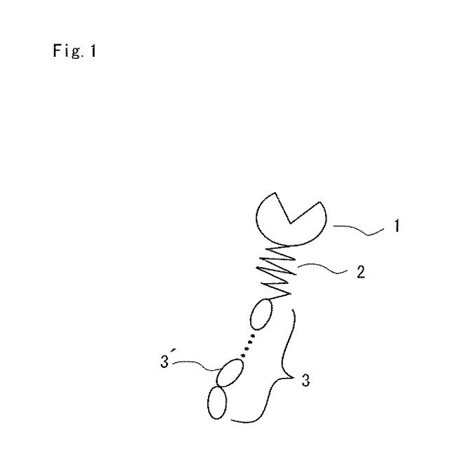

The chemical formula (13) shows a target recognition molecule of Example 1-1. In addition, FIG. 1 illustrates a conceptual structure of the target recognition molecule (molecule) of Example 1-1.

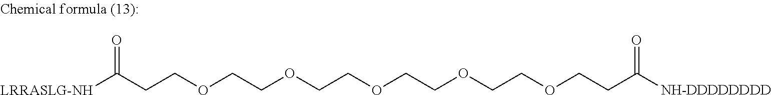

Referring to FIG. 1, the reference numeral 1 denotes a target recognition peptide segment; the reference numeral 2 denotes a connecting segment; the reference numeral 3 denotes an electrostatically-charged segment; and the reference numeral 3′ denotes a building block of the electrostatically-charged segment (amino acid residue in this example).

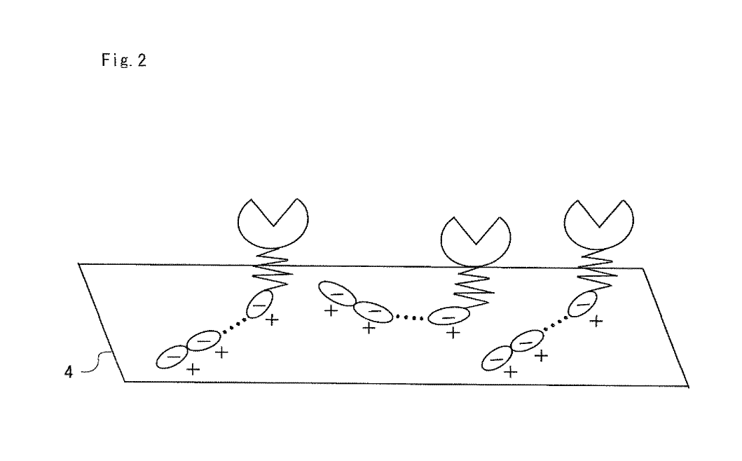

In addition, what is meant by “a series of dots” (i.e. “ . . . ” shown in the figure) is an omission of building blocks. The target recognition molecule is dissolved in a solution, and if the solution is at a pH from mildly acidic to alkaline, the electrostatically-charged segment portion becomes negatively charged. Therefore, upon contact of this solution with the surface of a positively charged electrode, the electrostatically-charged segment portion is electrically held and immobilized on the electrode surface. Referring to FIG. 2, there is shown an aspect of target recognition molecules being held on the electrode surface.

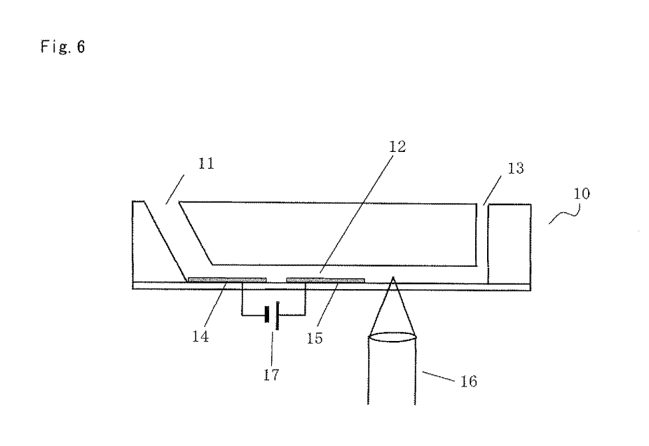

Making reference to FIG. 6, an example of how the target recognition molecule is used will be described. FIG. 6 illustrates an analyzer 10 which employs an analytical microchannel device, and the reference numeral 11 denotes a solution inlet; the reference numeral 12 denotes a channel; the reference numeral 13 denotes an outlet; the reference numerals 14 and 15 denote a pair of electrodes; and the reference numeral 16 denotes a detector. The basic procedure of an analytical method with the aid of this analyzer is as follows.

The target recognition molecule of Example 1-1 is dissolved in a carrier liquid composed of a phosphate buffered saline having a pH value of, for example, 7.3. The concentration is, for example, 100 ug/mL.

Next, with a direct current voltage (for example, from 1 to 10 V) impressed on the electrodes in pair (either one of the surfaces of the electrodes serves as an immobilization part), the target recognition molecule-containing carrier liquid is poured from the solution inlet 11 to flow through the inside of the channel 12. The target recognition molecule of Example 1-1 is attracted and immobilized onto the electrode 14 because the electrostatically-charged segment becomes negatively charged in the solution having a pH value of 7.3, as described above. In this state, the inside of the channel is cleansed by the aforesaid carrier liquid (containing no target recognition molecules). This completes an operation for immobilizing the target recognition molecule.

Since the target recognition molecule of Example 1-1 is characterized in that it can be electrically immobilized onto the electrode surface, thereby making it possible to provide switch control between immobilization and deimmobilization depending on the presence or absence of voltage application to the electrode. Therefore, it becomes possible to perform immobilization on the spot of assay. In addition, the inside of the channel is cleansed with no voltage applied, thereby making it possible to easily recycle the analytical chip.

In the target recognition molecule of Example 1-1, the connecting segment and the electrostatically-charged segment guarantee the degree of freedom of the target recognition peptide segment. Therefore, owing to immobilization on the electrode surface, the recognition performance with respect to a target substance will not be impaired, thereby making it possible to trap at high accuracy a target substance present in a test liquid.

The operation after immobilization may be based on a known analytical technique, e.g. a non-labeled immunoassay method or a labeled immunoassay method (for example, a sandwich assay method). In addition, it is possible to use, for example, a thermal lens, a surface plasmon resonance sensor, or a crystal oscillator as a detector and, in addition, it is also possible to use an electrode (immobilization part) itself as an electrochemical detector.

In addition, as a material to form the electrode 14, for example, metals such as gold (Au), copper (Cu), silver (Ag), titanium (Ti) et cetera or electrically conductive plastics can be used. And the electrode may be preformed, for example, by applying such a material to a site for immobilization during the preparation of an analytical chip.

### Example 1-2

According to a common procedure, a cysteine (C) is linked, as a base material immobilizing segment, to the C-terminal of the electrostatically-charged segment (amino acid sequence; DDDDDDDD) of the target recognition molecule of Example 1. The chemical formula (14) shows a target recognition molecule of Example 1-2.

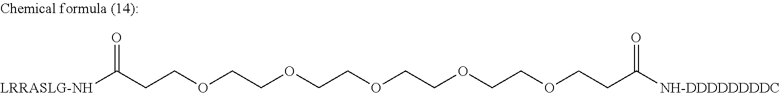

The target recognition molecule of Example 1-2 has such a property that it can be chemically linked, via the thiol group (elemental sulfur) of a cysteine residue, to the surface of the gold electrode. Therefore, with the gold electrode being electrically charged, a target recognition molecule containing solution is flowed, whereby target recognition molecules are densely brought together on the surface of the gold electrode and they are chemically linked to the surface of the gold (Au) electrode. After once chemically linked to the electrode surface, the immobilization state is retained even when the voltage application to the electrode is stopped.

### Example 1-3

In Example 1-2, a (N-[4-(p-Azidosalicylamido)butyl]-3′-(2′-pyridyldithio)propionamide) (APDP; produced by Thermo Corporation) was further reacted with the thiol group of the cysteine residue in order to introduce an azido group which is a photocrosslinking group into the terminal.

### (Synthetic Method)

A disulfide bond of the aforesaid APDP and an SH group of the cysteine are reacted (disulfide exchange) and linked together. The chemical formula (15) shows the structure of a target recognition molecule of Example 1-3.

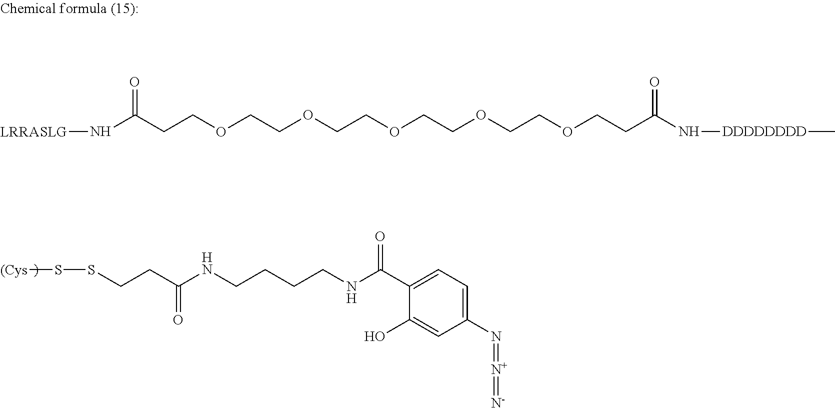

In the chemical formula (15) as shown above, the portion after this including a cysteine residue serves as a base material immobilizing segment. Further, in this example, it may be possible to arrange that, since the electrostatically-charged segment is composed of acidic amino acids and the cysteine is also an acidic amino acid, the cysteine-contained (DDDDDDDD-C) is made to serve as an electrostatically-charged segment while the portion after the S of the cysteine residue linked to the photocrosslinking group (azido group) is made to serve as a base material immobilizing segment.

Since, for the case of the target recognition molecule of Example 1-3, the base material immobilizing segment has a photocrosslinking group (azido group), this makes it possible to bring the target recognition molecule and the base material into chemical linkage (immobilization) by irradiation of the based material surface with light beams of UV long wavelength.

### Example 1-4

By use of an N-(6-Maleimidocaproyloxy)succinimide (Dojindo Laboratories) as a substitute for the (N-[4-(p-Azidosalicylamido)butyl]-3′-(2′-pyridyldithio)propionamide) of Example 1-3, a succinimide group was introduced into the thiol group of the cysteine residue.

A target recognition molecule according to this example has a succinimide group at its molecular end so that it can be brought into chemically linkage (immobilization) onto the base material surface having an amino group.

As a process for preparing, for example, the surface of a base material with amino groups, there is a method in which a thin film of gold is formed on a base plate and then a SAM film having an amino terminal is formed on the gold thin film by use of 11-Amino-1-undecanethiol, hydrochloride (Dojindo Laboratories).

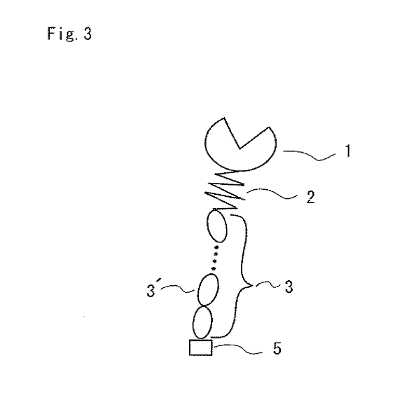

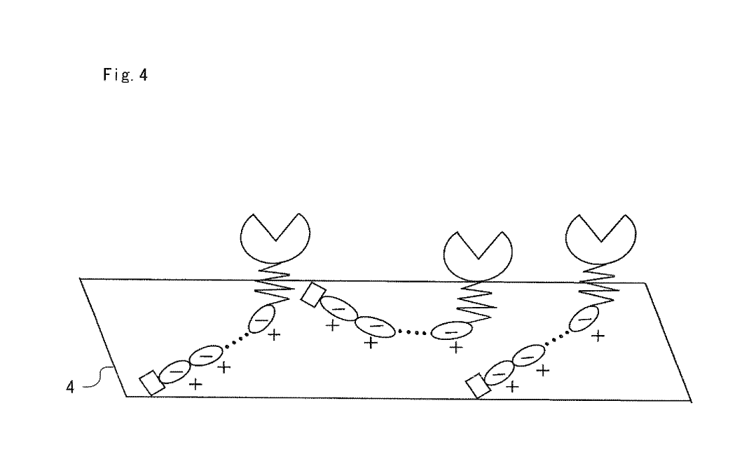

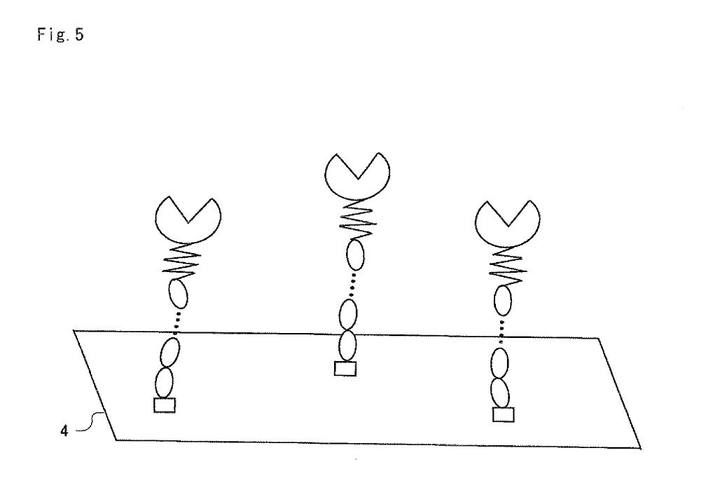

Referring to FIG. 3, there is shown a conceptual structure for the target recognition molecules of Examples 1-2 to 1-4. In addition, FIG. 4 shows an aspect of these target recognition molecules being electrostatically adsorbed and immobilized onto the electrically charged electrode surface (substrate surface). Further, FIG. 5 shows an aspect of the target recognition molecules being chemically linked, through their respective base material immobilizing segments, to the base material surface and uprising after stopping the voltage application to the electrode surface.

As shown in FIGS. 3-5, for the case of the target recognition molecules of Examples 1-2 to 1-4, molecules are brought together at the electrode by electrostatic attraction force and in this state, the functional group of each base material immobilizing segment can be linked to the electrode surface. It is therefore possible to accomplish high immobilization efficiency. In addition, after being chemically linked to the electrode surface, the immobilization state will be retained even when the electrical current to the electrode is disconnected, thereby achieving further improvement in usability.

### Second Group of Examples

### Example 2-1

As a target recognition peptide segment, there was used a segment (SEQ; LRRASLGC) resulting from linkage of a cysteine to the terminal of a PKA substrate peptide (SEQ: LRRASLG) of the same type as used in the aforesaid first group of examples.

On the other hand, as an electrostatically-charged segment, there was used a segment resulting from linkage of a cysteine (C) as a connecting segment to the N-terminal of a peptide (SEQ; RRRRRRRRRR) resulting from linking together ten arginines. The isoelectric point of this electrostatically-charged segment is 10.16 (average value).

As a material for a connecting segment, there was used a Mal-PEG-Mal whose number of polyethylene glycol building blocks, n, is 2, as shown in the following the chemical formula (16).

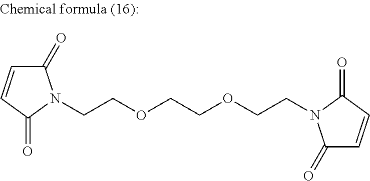

### (Synthetic Method)

Ten mM (100 times molar ratio) of Mal-PEG-Mal solution (containing 10% DMSO) was brought into reaction with 0.1 mM of the target recognition peptide segment. Thereafter, the uncrosslinked Mal-PEG-MaL was removed; 0.1 mM of the electrostatically-charged segment was brought into reaction; and the maleimide group of one end of the connecting segment and the SH group of a cysteine residue of the target recognition peptide segment were brought into linkage reaction while the maleimide group of the other end of the connecting segment and the SH group of a cysteine residue of the electrostatically-charged segment were brought into linkage reaction. In this way, a target recognition molecule of Example 2-1 as shown in chemical formula (17) was prepared.

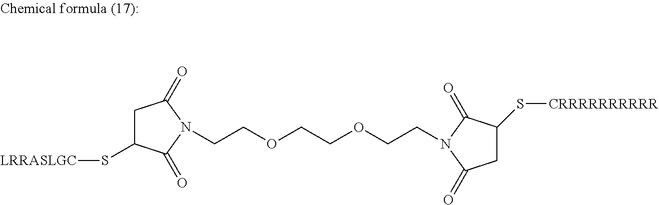

In addition, as shown in the following chemical formula (18), although the aforesaid chemical compound may be hydrolyzed in an aqueous solution in excess of pH value of 8 (it is possible to take the following two modes depending on the location of hydrolysis), the predetermined function of the present application will be exhibited even in such a case.

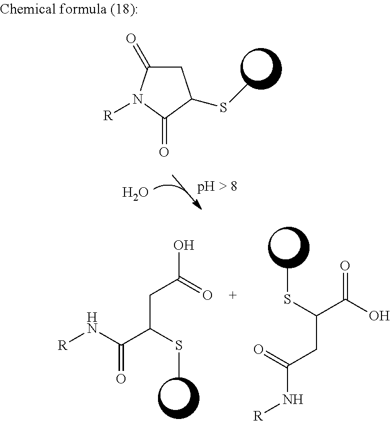

Since the target recognition molecule of Example 2-1 is used, with the electrostatically-charged segment being positively charged, it is preferred that the carrier solution is a solution at a pH from alkaline to acidic.

In addition, the reason for the introduction of cysteine is to cause the linkage position of the peptide segment to the connecting segment and the linkage position of the connecting segment to the electrostatically-charged segment to be terminal. Alternatively, it may be arranged such that, without the introduction of cysteine, these linkage positions are made to be terminal by modification of a reactive functional group (for example, an amino group).

In addition, as a constituent amino acid of the aforesaid electrostatically-charged segment, either lysine which is a basic amino acid may be used in place of arginine, or both of lysine and arginine may be used.

### Example 2-2

As in Example 1-2 described above, a cysteine (C) as a base material immobilizing segment was linked to the C-terminal of an electrostatically-charged segment (amino acid sequence; RRRRRRRRRR) of a target recognition molecule. A structure of the target recognition molecule of Example 2-2 is shown in the chemical formula (19).

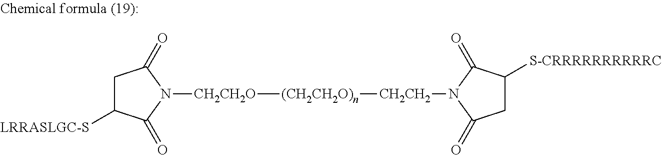

### Example 2-3

As in Example 1-3 described above, there was prepared a target recognition molecule according to Example 2-3 in which a photocrosslinking group (an azido group) was introduced into the molecular end. The target recognition molecule of Example 2-3 is shown in the chemical formula (20).

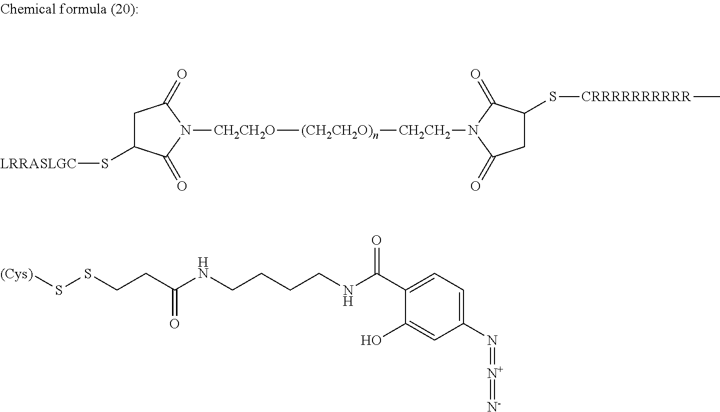

### Example 2-4

As in Example 1-4 described above, there was prepared a target recognition molecule according to Example 2-4 in which a succinimide group was introduced into the thiol group of a cysteine residue. A structure of the target recognition molecule of Example 2-4 is shown in the chemical formula (21). Note that the letter “X” indicates a connecting segment and the letter “P” indicates a peptide.

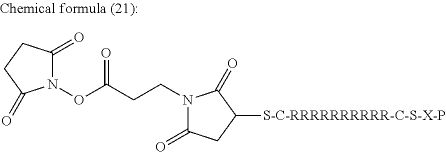

### Third Group of Examples

### Example 3

As a target recognition peptide segment, there was used a PKA substrate peptide (SEQ; LRRASLG) of the same type as used in the forgoing first group of examples.

As an electrostatically-charged segment, there was used a segment that has a polyacrylic acid building block (n=14, R=Na) as shown in the following chemical formula (1).

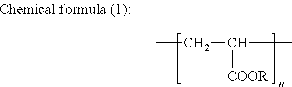

wherein R is H, Na, or K.

As a connecting segment, there was used an NHS-PEG2-OH having two polyethylene glycol building blocks (n=2). The OH group of this chemical compound and the carboxyl group of the electrostatically-charged segment were brought into ester linkage, and the succinimide group and the amino group of the target recognition peptide segment were linked together.

A structure of the target recognition molecule of Example 3 is shown in the chemical formula (22).

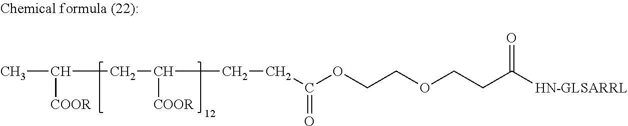

wherein R is H, Na, or K.

### Fourth Group of Examples

### Example 4

As a target recognition peptide segment, there was used a PKA substrate peptide (SEQ: LRRASLG) of the same type as used in the foregoing first group of examples.

As an electrostatically-charged segment, there was used a segment that has polyethylenimine building blocks (n=14) as shown in the following the chemical formula (7).

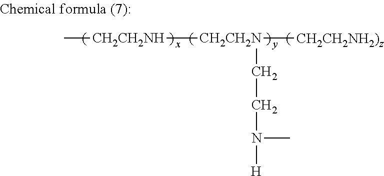

wherein x:y:x=0.5:0.25:0.25 and [x+y+z] is an integer not less than 3 nor more than 150.

As a connecting segment, there was used a Bis(NHS)PEG₅ [Bis-N-Succinimidyl-(diethyene-glycol)ester] containing five polyethylene glycol building blocks (n=5).

A structure of the target recognition molecule of Example 4 is shown in chemical formula 23.

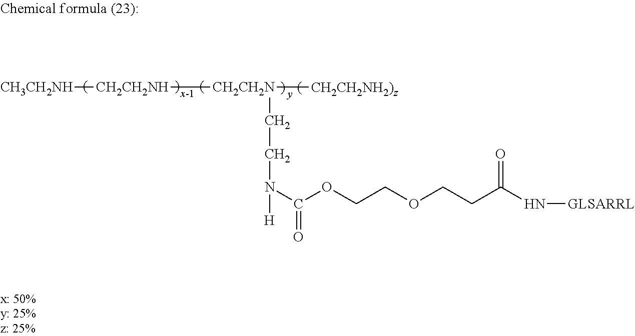

### Fifth Group of Examples

### Example 5

As a target recognition peptide segment, there was used a PKA substrate peptide (SEQ: LRRASLG) of the same type as used in the foregoing first group of examples.

As an electrostatically-charged segment, there was used a segment that has poly-diallyldimethylammonium chloride building blocks (n=14) shown in the chemical formula (9). In addition, a polyacrylic acid building block for linkage to the connecting segment was introduced into the electrostatically-charged segment.

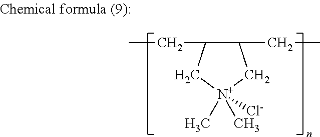

As a connecting segment, there was used an NHS-PEG2-OH having two polyethylene glycol building blocks (n=2). The OH group of this chemical compound and the carboxyl group introduced into the electrostatically-charged segment were brought into ester, linkage and the succinimide group and the amino group of the target recognition peptide segment were linked together.

A structure of the target recognition molecule of Example 5 is shown in the chemical formula (24).

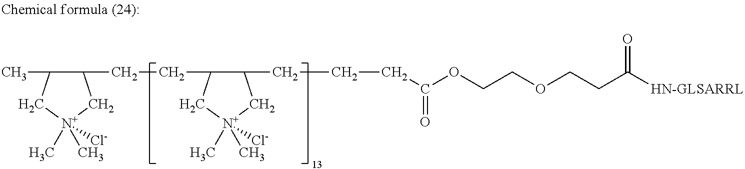

### Sixth Group of Examples

### Example 6

In Example 5, as an electrostatically-charged segment, there was used a segment that has, as a substitute for poly-diallyldimethylammonium chloride, polyallylamine building blocks (n=14) shown in the chemical formula (8). In addition, a polyacrylic acid building block for linkage to the connecting segment was introduced into the electrostatically-charged segment. With this exception, a target recognition molecule according to Example 6 was prepared in the same way as Example 5. The structure of this molecule is shown in the chemical formula (25).

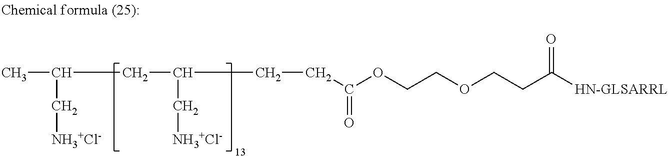

### Seventh Group of Examples

### Example 7

As a target recognition peptide segment, there was used a PKA substrate peptide (SEQ: LRRASLG) of the same type as used in the foregoing first group of examples.

As an electrostatically-charged segment, there was used a segment that has polyvinylpyridine building blocks (n=14) shown in the chemical formula (10). In addition, a polyacrylic acid building block for linkage to the connecting segment was introduced into the electrostatically-charged segment.

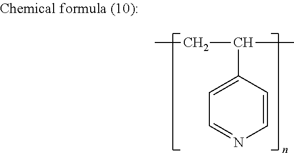

As a connecting segment, there was used an NHS-PEG2-OH having two polyethylene glycol building blocks (n=2). The OH group of this chemical compound and the carboxyl group introduced into the electrostatically-charged segment were brought into ester linkage and the succinimide group and the amino group of the target recognition peptide segment were linked together.

A structure of the target recognition molecule of Example 7 is shown in the chemical formula (26).

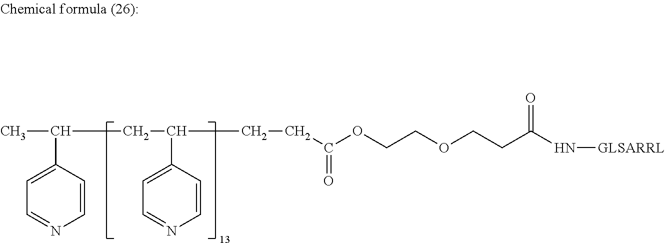

### Eighth Group of Examples

### Example 8

As a target recognition peptide segment, there was used a PKA substrate peptide (SEQ: LRRASLG) of the same type as used in the foregoing first group of examples.

As an electrostatically-charged segment, there was used a segment that has dextran sulfate building blocks (n=14; sulfonation rate (the ratio of SO₃Na account for R): 30%) shown in the chemical formula (4). In addition, note that it suffices if the sulfonation rate is 10% or higher.

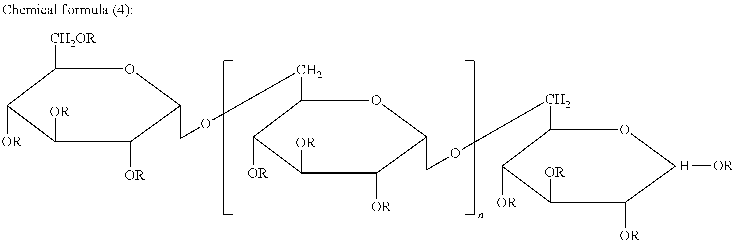

wherein R is SO₃Na or H.

As a connecting segment, there was used an NHS-PEG2-OH having two polyethylene glycol building blocks (n=2). The OH group of this chemical compound and the hydroxysulfonyl group (SO₃H group) of the electrostatically-charged segment were brought into ester linkage and the succinimide group and the amino group of the target recognition peptide segment were linked together.

A structure of the target recognition molecule of Example 8 is shown in the chemical formula (27).

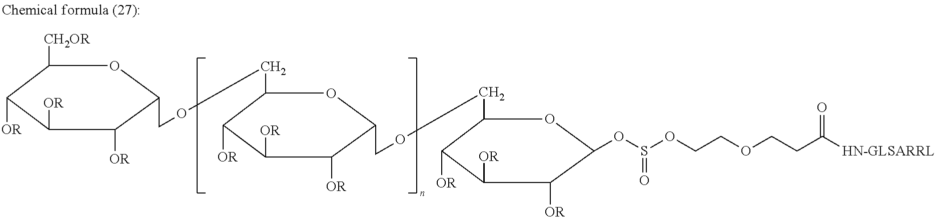

wherein R is SO₃Na or H.

### Ninth Group of Examples

### Example 9

As a target recognition peptide segment, there was used a PKA substrate peptide (SEQ: LRRASLG) of the same type as used in the foregoing first group of examples.

As an electrostatically-charged segment, there was used a segment that has chondroitin sulfate building blocks (n=14, R═H) shown in the chemical formula (5).

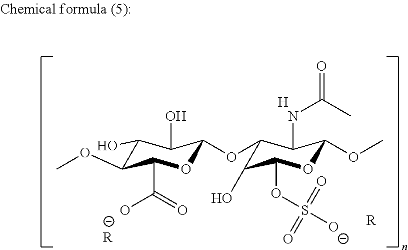

wherein R is H, Na, or K.

As a connecting segment, there was used an NHS-PEG2-OH having two polyethylene glycol building blocks (n=2). The OH group of this chemical compound and the hydroxysulfonyl group (SO₃H group) of the electrostatically-charged segment were brought into ester linkage and the succinimide group and the amino group of the target recognition peptide segment were linked together.

A structure of the target recognition molecule of Example 9 is shown in the chemical formula (28).

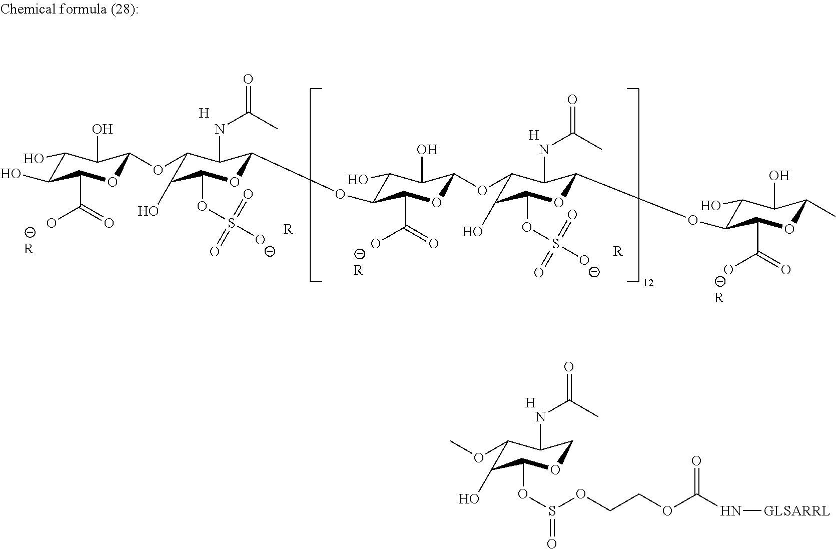

wherein R is H, Na, or K.

### Tenth Group of Examples

### Example 10-1

As a target recognition peptide segment, there was used a segment resulting from introducing a cysteine C into the end of a PKA substrate peptide (SEQ: LRRASLG) of the same type as used in the foregoing first group of examples.

As an electrostatically-charged segment, there was used a segment that has an octonucleotide (one chain of which is a poly-deoxyadenosine-monophosphate and the other chain (complementary) of which is a poly-deoxythymidine-monophosphate) with a (CH₂)₆SH introduced into a 5′-terminal phosphoric acid of the one chain (see the chemical formula (29)).

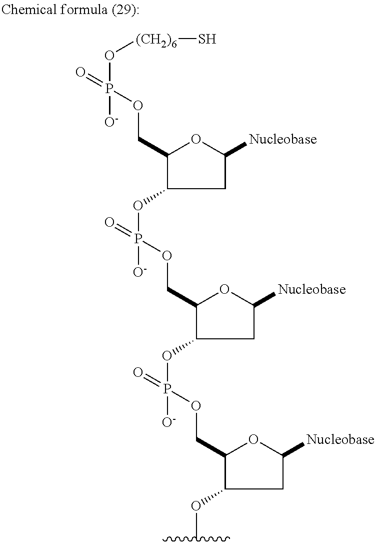

### (Connecting Segment)

As a connecting segment, there was used a Bis Maleimidoethane (Thermo Corporation) as defined in the general chemical formula (30). The maleimide group of one end thereof was reacted with the SH group of the cysteine residue in the target recognition peptide segment while the maleimide group of the other end was linked to the SH group of the electrostatically-charged segment.

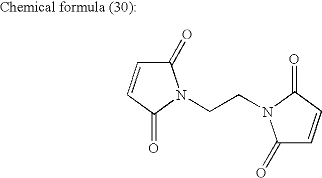

### (Synthetic Method)

Ten mM (a 1:100 molar ratio) of Bis Maleimidoethane (containing 10% DMSO) was brought into reaction with 0.1 mM of the target recognition peptide segment. Thereafter, the uncrosslinked Bis Maleimidoethane was removed; 0.1 mM of the electrostatically-charged segment was brought into reaction; and the maleimide group of one end of the connecting segment and the SH group of a cysteine residue of the target recognition peptide segment were brought into linkage reaction while the maleimide group of the other end of the connecting segment and the SH group of the electrostatically-charged segment were brought into linkage reaction.

### Example 10-2

As a target recognition peptide segment, there was used a segment resulting from introduction of a cysteine (C) into the terminal of a PKA substrate peptide (SEQ; LRRASLG) of the same type as used in Example 10-1.

As an electrostatically-charged segment, there was used a DNA of the same type as used in Example 10-1.

### (Connecting Segment)

As a connecting segment, there was employed 1,4-Di-[3′-(2-pyridyldithio)-propionamido]butane (Thermo Corporation) as shown in the general chemical formula (31). One of the disulfides was reacted with the SH group of a cysteine residue of the foregoing target recognition peptide segment while the other disulfide was linked to the SH group of the electrostatically-charged segment.

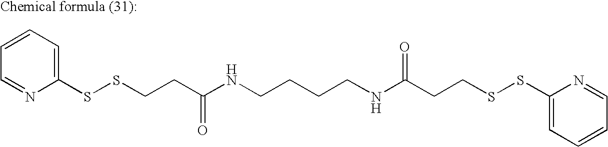

### Example 10-3

As a target recognition peptide segment, there was used a segment resulting from introduction of a cysteine (C) into the terminal of a PKA substrate peptide (SEQ; LRRASLG) of the same type as used in Example 10-1.

As an electrostatically-charged segment, there was used a DNA of the same type as used in Example 10-1.

### (Connecting Segment)

As a connecting segment, there was employed 1,11-Bis-maleimido-triethyleneglycol (Thermo Corporation) as shown in the general the chemical formula (32). The maleimide group of one end thereof was reacted with the SH group of a cysteine residue of the target recognition peptide segment while the maleimide group of the other end was linked to the SH group of the electrostatically-charged segment.

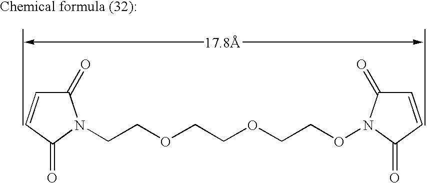

### Example 10-4

As a target recognition peptide segment, there was used a segment resulting from introduction of a cysteine (C) into the terminal of a PKA substrate peptide (SEQ; LRRASLG) of the same type as used in Example 10-1.

As an electrostatically-charged segment, there was used a DNA of the same type as used in Example 10-1, with (CH₂)₆SH further introduced to a 5′-terminal phosphoric acid of the other (complementary) chain.

### (Connecting Segment)

As a connecting segment, there was employed a 1,11-Bis-maleimido-triethyleneglycol (Thermo Corporation) of the same type as used in Example 10-3. The maleimide group of one end thereof was reacted with the SH group of a cysteine residue of the target recognition peptide segment while the maleimide group of the other end was linked to the SH group of the electrostatically-charged segment.

### (Synthetic Method)

Ten mM (a 1:100 molar ratio) of Bis Maleimidoethane (containing 10% DMSO) was brought into reaction with 0.1 mM of the target recognition peptide segment. Thereafter, the uncrosslinked Bis Maleimidoethane was removed; 0.1 mM of the electrostatically-charged segment was brought into reaction; and the maleimide group of one end of the connecting segment and the SH group of a cysteine residue of the target recognition peptide segment were brought into linkage reaction while the maleimide group of the other end of the connecting segment and the SH group of the electrostatically-charged segment were brought into linkage reaction. A 10 mMN-[g-Maleimidobutyryloxy]succinimide ester solution as shown in the following chemical formula (33) is mixed for reaction with the SH group of the electrostatically-charged segment, and a base material immobilizing segment having a succinimide group at the terminal is introduced.

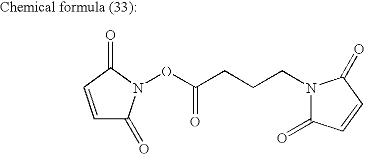

### Supplementary Information

Each of the foregoing examples uses, as a target recognition peptide segment, a protein kinase A substrate peptide. However, the target recognition peptide segment as a main element of the present invention is not limited to the aforesaid substance. The target recognition peptide segment according to the present invention may be any peptide as long as it can specifically recognize a target substance. Whether or not it is a peptide that specifically recognizes a target substance is determined in relation to a target substance as a detection object. More specifically, known technologies, such as pharge display technology (Pharge Display—Laboratory Manual. Cold Spring Harbor Laboratory Press, 2001 Barbas. C. et al.) and spot synthesis technology (The SPOT-synthesis technique. Synthesis peptide arrays on membrane supports-principles and applications. J. Immunol. Methods 267 2002 13-26 R. Frank), are used to decide a peptide sequence capable of recognition of a target substance as a detection object, and a peptide with such a sequence is selected as a target recognition peptide segment.

In addition, the material of the peptide of the target recognition peptide segment may be either naturally derived or artificially synthesized, and there is no limitation regarding the process of peptide synthesis. As a process of peptide synthesis, there are, for example, a solid-phase synthetic method, a liquid-phase synthetic method, and a process that employs gene expression.

In addition, in Example 3, it is possible to use, in place of an electrostatically-charged segment having a polyacrylic acid building block as described above, an electrostatically-charged segment having either a polystyrene sulfonic acid building block as shown in the chemical formula (2) or a polyvinyl sulfate building block as shown in the chemical formula (3).

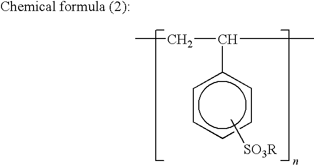

wherein R is H, Na, or K.

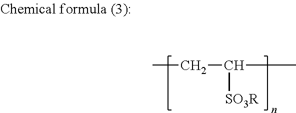

wherein R is H, Na, or K.

Here, if the length (arm length) of the electrostatically-charged segment is too long, this causes disadvantages such as an intermolecular entanglement. On the other hand, if the length of the electrostatically-charged segment is too short, this results in a reduced degree of freedom of the target recognition segment. Therefore, it is required that the length of the electrostatically-charged segment be properly selected in relation to its own properties as well as in relation to the target recognition segment. Therefore, preferably, the length of the electrostatically-charged segment exceeds the length of the target recognition peptide segment. It is more preferable to select a repeat unit (n) so that the length of the electrostatically-charged segment is from once to twice the length of the target recognition peptide segment. In addition, generally, if the repeat unit (n) is less than 3, this is undesirable because the force of attraction by electrostatic interaction becomes deficient. On the other hand, if the repeat unit (n) exceeds 150, this is also undesirable because of, for example, the synthesis cost increases and there occurs a molecular entanglement.

In addition, as a carrier solution for an analytical chip using a target recognition molecule, there is usually used an aqueous solution having a near-neutral pH value (pH value=about 7±1). However, since the average isoelectric point of each of the target recognition peptide segments of the foregoing examples is 7.3, the electric charge of their target recognition peptide segment part reaches a negligible level if the target recognition molecule according to each of the examples is solved in a neutral carrier solution (pH value=about 7±1). In other words, in the target recognition molecule according to each of the examples, even in the case where the electrostatically-charged functional group of the electrostatically-charged segment is one that is electrically charged to whatever type of polarity, namely either positively or negatively, there is very little influence on the electric charge of the target recognition peptide segment part. Therefore, there is no need to specify the type of electric charge of the electrostatically-charged segment in relation to the average isoelectric point of the target recognition peptide segment.

On the other hand, if the average isoelectric point of the target recognition peptide segments is 6 or less or is 8 or greater, this increases the influence of the electric charge of the target recognition peptide segment accounting for the entire target recognition peptide segment. Therefore, if the average isoelectric point of the target recognition peptide segment is 6 or less, it is preferred that the electrostatically-charged functional group of the electrostatically-charged segment is a functional group that becomes negatively electrically charged in an aqueous solution having a pH value of 7 or greater. If a target recognition molecule that meets such a requirement is dissolved in a carrier aqueous solution having a pH value of 7 or greater (for example, pH value=7.8), the negative charge density of the electrostatically-charged segment increases to a sufficient level, whereby such target recognition molecules can be efficiently brought together in a positively electrically charged immobilization site (electrode) and reversibly immobilized there.

On the other hand, if the average isoelectric point of the target recognition peptide segment is 8 or greater, it is preferred that the electrostatically-charged functional group of the electrostatically-charged segment is a functional group that becomes positively electrically charged in an aqueous solution having a pH value of 7 or less. If a target recognition molecule that meets such a requirement is dissolved in a carrier aqueous solution having a pH value of 7 or less (for example, pH value=6.2), the positive charge density of the electrostatically-charged segment increases to a sufficient level. Therefore, by meeting this requirement, such target recognition molecules can be efficiently brought together in a negatively electrically charged immobilization site (electrode) and reversibly immobilized there.

In the foregoing examples 3-9, there are shown target recognition molecules not provided with a base material immobilizing segment. However, it is possible to chemically link a base material immobilizing segment capable of covalent linkage to a base material with these molecules, as shown in the first group of examples.

### INDUSTRIAL APPLICABILITY

The target recognition molecule of the present invention is a novel chemical molecule including a target recognition segment as a binding site which specifically interacts with a target substance and an electrostatically-charged segment which is provided with an electrostatic property. The use of a solution containing a target recognition molecule of the present invention makes it possible that such target recognition molecules can be densely brought together in a charge-applied immobilization site in a self-assembly manner and reversibly immobilized there. In addition, the use of a target recognition molecule of the present invention which is provided with a base material immobilizing segment makes it possible that such target recognition molecules can be densely brought together in a charge-applied immobilization site in a self-assembly manner and reversibly immobilized there. These target recognition molecules of the present invention contribute to considerable improvement in the usability, assay accuracy, and reliability of analytical devices including an analytical chip and other like device. Therefore, the industrial applicability of the target recognition molecules of the present invention is high.

### REFERENCE SIGNS LIST

1 Target recognition segment
2 Connecting segment
3 Electrostatically-charged segment
3′ Electrostatically-charged segment repeat unit
4 Base material
5 Base material immobilizing segment
10 Analytical chip
11 Liquid inlet port
12 Microchannel
13 Outlet port
14, 15 Electrodes (Either one of them serves as an immobilization site.)
16 Detector
17 Power supply

What is claimed is:

1. A target recognition molecule comprising:
a target recognition peptide segment as a specific binding site for a target substance;
an electrostatically-charged segment which is provided with electrostatically-charged functional groups capable of being electrically charged with charges of the same polarity in the same solution; and
a connecting segment which chemically links with said target recognition peptide segment and with said electrostatically-charged segment for establishing a connection between both said segments.

2. The target recognition molecule as set forth in claim 1,
wherein said electrostatically-charged segment comprises three or more electrostatically-charged functional groups capable of being electrically charged with charges of the same polarity in the same solution.

3. The target recognition molecule as set forth in claim 2,
wherein the isoelectric point of said target recognition peptide segment is 6 or less; and
wherein said electrostatically-charged functional groups of said electrostatically-charged segment are functional groups which become negatively electrically charged in an aqueous solution of a pH value of 7 or greater.

4. The target recognition molecule as set forth in claim 3,
wherein said target recognition peptide segment contains a cysteine residue, and wherein said connecting segment is chemically linked to elemental sulfur of said cysteine residue.

5. The target recognition molecule as set forth in claim 3,
wherein one end of said target recognition peptide segment is a cysteine residue, and wherein said connecting segment is chemically linked to elemental sulfur of said cysteine residue.

6. The target recognition molecule as set forth in claim 3,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

7. The target recognition molecule as set forth in claim 6,
wherein said connecting segment has no functional groups that become electrically charged in the solution.

8. The target recognition molecule as set forth in claim 7,
wherein said electrostatically-charged segment has no functional groups that become electrically charged to different polarities in the same solution.

9. The target recognition molecule as set forth in claim 8,
wherein said target recognition peptide segment comprises a peptide which has not less than 3 nor more than 19 amino acid residues.

10. The target recognition molecule as set forth in claim 9,
wherein further chemically linked to said electrostatically-charged segment is a base material immobilizing segment which is provided with a functional group for linkage to a base material.

11. The target recognition molecule as set forth in claim 10,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

12. The target recognition molecule as set forth in claim 2,
wherein the isoelectric point of said target recognition peptide segment is 8 or greater; and
wherein said electrostatically-charged functional groups of said electrostatically-charged segment are functional groups which become positively electrically charged in an aqueous solution of a pH value of 7 or less.

13. The target recognition molecule as set forth in claim 12,
wherein said target recognition peptide segment contains a cysteine residue, and wherein said connecting segment is chemically linked to elemental sulfur of said cysteine residue.

14. The target recognition molecule as set forth in claim 12,
wherein one end of said target recognition peptide segment is a cysteine residue, and wherein said connecting segment is chemically linked to elemental sulfur of said cysteine residue.

15. The target recognition molecule as set forth in claim 12,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

16. The target recognition molecule as set forth in claim 15,
wherein said connecting segment has no functional groups that become electrically charged in the solution.

17. The target recognition molecule as set forth in claim 16,
wherein said electrostatically-charged segment has no functional groups that become electrically charged to different polarities in the same solution.

18. The target recognition molecule as set forth in claim 17,
wherein said target recognition peptide segment comprises a peptide which has not less than 3 nor more than 19 amino acid residues.

19. The target recognition molecule as set forth in claim 18,
wherein further chemically linked to said electrostatically-charged segment is a base material immobilizing segment which is provided with a functional group for linkage to a base material.

20. The target recognition molecule as set forth in claim 19,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

21. The target recognition molecule as set forth in claim 3,
wherein said electrostatically-charged segment comprises a peptide which has 3 or more amino acid residues.

22. The target recognition molecule as set forth in claim 9,
wherein said peptide as said electrostatically-charged segment contains three or more acidic amino acid residues, one or more of which are selected from a group composed of aspartic acid and glutamic acid, and contains neither an arginine residue nor a lysine residue.

23. The target recognition molecule as set forth in claim 22,
wherein said target recognition peptide segment contains a cysteine residue, and wherein said connecting segment is chemically linked to elemental sulfur of said cysteine residue.

24. The target recognition molecule as set forth in claim 22,
wherein one end of said target recognition peptide segment is a cysteine residue, and wherein said connecting segment is chemically linked to elemental sulfur of said cysteine residue.

25. The target recognition molecule as set forth in claim 22,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

26. The target recognition molecule as set forth in claim 25,
wherein said connecting segment has no functional groups that become electrically charged in the solution.

27. The target recognition molecule as set forth in claim 26,
wherein said target recognition peptide segment comprises a peptide which has not less than 3 nor more than 19 amino acid residues.

28. The target recognition molecule as set forth in claim 27,
wherein the number of amino acid residues forming said electrostatically-charged segment is larger than the number of amino acid residues forming said target recognition peptide segment.

29. The target recognition molecule as set forth in claim 28,
wherein further chemically linked to said electrostatically-charged segment is a base material immobilizing segment which is provided with a functional group for linkage to a base material.

30. The target recognition molecule as set forth in claim 29,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

31. The target recognition molecule as set forth in claim 4,
wherein said electrostatically-charged segment comprises a peptide which has 3 or more amino acid residues.

32. The target recognition molecule as set forth in claim 31,
wherein said peptide as said electrostatically-charged segment contains three or more basic amino acid residues, one or more of which are selected from a group composed of arginine and lysine, and contains neither an aspartic acid residue nor a glutamic acid residue.

33. The target recognition molecule as set forth in claim 32,
wherein said target recognition peptide segment contains a cysteine residue, and wherein said connecting segment is chemically linked to elemental sulfur of said cysteine residue.

34. The target recognition molecule as set forth in claim 32,
wherein one end of said target recognition peptide segment is a cysteine residue, and wherein said connecting segment is chemically linked to elemental sulfur of said cysteine residue.

35. The target recognition molecule as set forth in claim 32,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

36. The target recognition molecule as set forth in claim 35,
wherein said connecting segment has no functional groups that become electrically charged in the solution.

37. The target recognition molecule as set forth in claim 36,
wherein said target recognition peptide segment comprises a peptide which has not less than 3 nor more than 19 amino acid residues.

38. The target recognition molecule as set forth in claim 37,
wherein the number of amino acid residues forming said electrostatically-charged segment is larger than the number of amino acid residues forming said target recognition peptide segment.

39. The target recognition molecule as set forth in claim 38,
wherein further chemically linked to said electrostatically-charged segment is a base material immobilizing segment which is provided with a functional group for linkage to a base material.

40. The target recognition molecule as set forth in claim 39,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

41. The target recognition molecule as set forth in claim 1,
wherein said electrostatically-charged segment is a segment which has a polyacrylic acid building block represented by the following chemical formula (1) with n being not less than 3 nor more than 150. 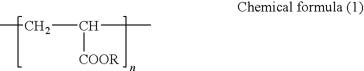
wherein R is H, Na, or K.

42. The target recognition molecule as set forth in claim 41,
wherein said target recognition peptide segment contains a cysteine residue, and wherein said connecting segment is chemically linked to elemental sulfur of said cysteine residue.

43. The target recognition molecule as set forth in claim 41,
wherein one end of said target recognition peptide segment is a cysteine residue, and wherein said connecting segment is chemically linked to elemental sulfur of said cysteine residue.

44. The target recognition molecule as set forth in claim 41,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

45. The target recognition molecule as set forth in claim 44,
wherein said target recognition peptide segment comprises a peptide which has not less than 3 nor more than 19 amino acid residues.

46. The target recognition molecule as set forth in claim 45,
wherein further chemically linked to said electrostatically-charged segment is a base material immobilizing segment which is provided with a functional group for linkage to a base material.

47. The target recognition molecule as set forth in claim 46,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

48. The target recognition molecule as set forth in claim 1,
wherein said electrostatically-charged segment is a segment having a polystyrene sulfonic acid building block represented by the following chemical formula (2) with n being not less than 3 nor more than 150. 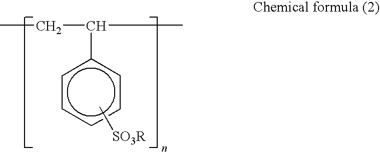
wherein R is H, Na, or K.

49. The target recognition molecule as set forth in claim 48,
wherein said target recognition peptide segment contains a cysteine residue, and wherein said connecting segment is chemically linked to elemental sulfur of said cysteine residue.

50. The target recognition molecule as set forth in claim 48,
wherein one end of said target recognition peptide segment is a cysteine residue, and wherein said connecting segment is chemically linked to elemental sulfur of said cysteine residue.

51. The target recognition molecule as set forth in claim 48,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

52. The target recognition molecule as set forth in claim 51,
wherein said target recognition peptide segment comprises a peptide which has not less than 3 nor more than 19 amino acid residues.

53. The target recognition molecule as set forth in claim 52,
wherein further chemically linked to said electrostatically-charged segment is a base material immobilizing segment which is provided with a functional group for linkage to a base material.

54. The target recognition molecule as set forth in claim 53,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

55. The target recognition molecule as set forth in claim 1,
wherein said electrostatically-charged segment is a segment having a polyvinyl sulfate building block represented by the following chemical formula (3) with n being not less than 3 nor more than 150. 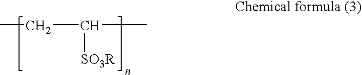
wherein R is H, Na, or K.

56. The target recognition molecule as set forth in claim 55,
wherein said target recognition peptide segment contains a cysteine residue, and wherein said connecting segment is chemically linked to elemental sulfur of said cysteine residue.

57. The target recognition molecule as set forth in claim 55,
wherein one end of said target recognition peptide segment is a cysteine residue, and wherein said connecting segment is chemically linked to elemental sulfur of said cysteine residue.

58. The target recognition molecule as set forth in claim 55,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

59. The target recognition molecule as set forth in claim 58,
wherein said target recognition peptide segment comprises a peptide which has not less than 3 nor more than 19 amino acid residues.

60. The target recognition molecule as set forth in claim 59,
wherein further chemically linked to said electrostatically-charged segment is a base material immobilizing segment which is provided with a functional group for linkage to a base material.

61. The target recognition molecule as set forth in claim 60,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

62. The target recognition molecule as set forth in claim 1,
wherein said electrostatically-charged segment is a segment having a dextran sulfate building block represented by the following chemical formula (4) with n being not less than 1 nor more than 150. 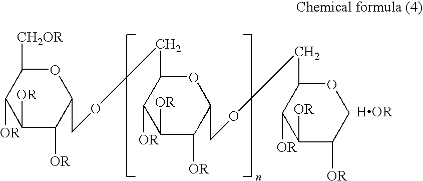
wherein R is SO₃Na or H.

63. The target recognition molecule as set forth in claim 62,
wherein said target recognition peptide segment contains a cysteine residue, and wherein said connecting segment is chemically linked to elemental sulfur of said cysteine residue.

64. The target recognition molecule as set forth in claim 63,
wherein one end of said target recognition peptide segment is a cysteine residue, and wherein said connecting segment is chemically linked to elemental sulfur of said cysteine residue.

65. The target recognition molecule as set forth in claim 62,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

66. The target recognition molecule as set forth in claim 65,
wherein said target recognition peptide segment comprises a peptide which has not less than 3 nor more than 19 amino acid residues.

67. The target recognition molecule as set forth in claim 66,
wherein further chemically linked to said electrostatically-charged segment is a base material immobilizing segment which is provided with a functional group for linkage to a base material.

68. The target recognition molecule as set forth in claim 67,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

69. The target recognition molecule as set forth in claim 1,
wherein said electrostatically-charged segment is a segment having a chondroitin sulfate building block represented by the following chemical formula (5) with n being not less than 1 nor more than 150. 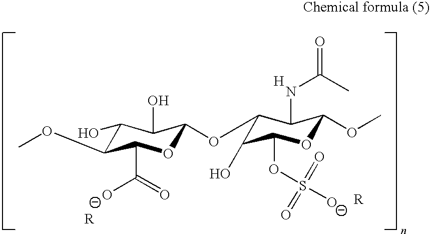
wherein R is H, Na, or K.

70. The target recognition molecule as set forth in claim 69,
wherein said target recognition peptide segment contains a cysteine residue, and wherein said connecting segment is chemically linked to elemental sulfur of said cysteine residue.

71. The target recognition molecule as set forth in claim 69,
wherein one end of said target recognition peptide segment is a cysteine residue, and wherein said connecting segment is chemically linked to elemental sulfur of said cysteine residue.

72. The target recognition molecule as set forth in claim 69,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

73. The target recognition molecule as set forth in claim 72,
wherein said target recognition peptide segment comprises a peptide which has not less than 3 nor more than 19 amino acid residues.

74. The target recognition molecule as set forth in claim 73,
wherein further chemically linked to said electrostatically-charged segment is a base material immobilizing segment which is provided with a functional group for linkage to a base material.

75. The target recognition molecule as set forth in claim 74,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

76. The target recognition molecule as set forth in claim 1,
wherein said electrostatically-charged segment is a segment having a nucleotide building block represented by the following chemical formula (6) with n being not less than 3 nor more than 150. 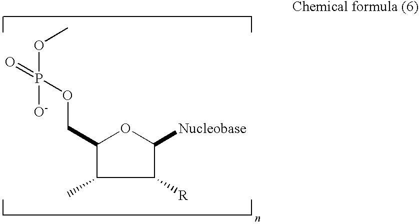
wherein R is H or OH.

77. The target recognition molecule as set forth in claim 76,
wherein said target recognition peptide segment contains a cysteine residue, and wherein said connecting segment is chemically linked to elemental sulfur of said cysteine residue.

78. The target recognition molecule as set forth in claim 76,
wherein one end of said target recognition peptide segment is a cysteine residue, and wherein said connecting segment is chemically linked to elemental sulfur of said cysteine residue.

79. The target recognition molecule as set forth in claim 76,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

80. The target recognition molecule as set forth in claim 79,
wherein said target recognition peptide segment comprises a peptide which has not less than 3 nor more than 19 amino acid residues.

81. The target recognition molecule as set forth in claim 80,
wherein further chemically linked to said electrostatically-charged segment is a base material immobilizing segment which is provided with a functional group for linkage to a base material.

82. The target recognition molecule as set forth in claim 81,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

83. The target recognition molecule as set forth in claim 1,
wherein said electrostatically-charged segment is a segment having a polyethylenimine building block represented by the following chemical formula (7), 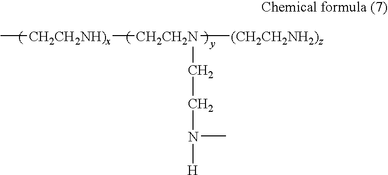
wherein x:y:x=0.5:0.25:0.25 and [x+y+z] is an integer not less than 3 nor more than 150.

84. The target recognition molecule as set forth in claim 83,
wherein said target recognition peptide segment contains a cysteine residue, and wherein said connecting segment is chemically linked to elemental sulfur of said cysteine residue.

85. The target recognition molecule as set forth in claim 83,
wherein one end of said target recognition peptide segment is a cysteine residue, and wherein said connecting segment is chemically linked to elemental sulfur of said cysteine residue.

86. The target recognition molecule as set forth in claim 83,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

87. The target recognition molecule as set forth in claim 86,
wherein said target recognition peptide segment comprises a peptide which has not less than 3 nor more than 19 amino acid residues.

88. The target recognition molecule as set forth in claim 87,
wherein further chemically linked to said electrostatically-charged segment is a base material immobilizing segment which is provided with a functional group for linkage to a base material.

89. The target recognition molecule as set forth in claim 88,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

90. The target recognition molecule as set forth in claim 1,
wherein said electrostatically-charged segment is a segment having a polyallylamine hydrochloride building block represented by the following chemical formula (8) with n being not less than 3 nor more than 150.

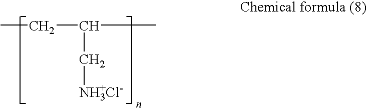

91. The target recognition molecule as set forth in claim 90,
wherein said target recognition peptide segment contains a cysteine residue, and wherein said connecting segment is chemically linked to elemental sulfur of said cysteine residue.

92. The target recognition molecule as set forth in claim 90,
wherein one end of said target recognition peptide segment is a cysteine residue, and wherein said connecting segment is chemically linked to elemental sulfur of said cysteine residue.

93. The target recognition molecule as set forth in claim 90,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

94. The target recognition molecule as set forth in claim 93,
wherein said target recognition peptide segment comprises a peptide which has not less than 3 nor more than 19 amino acid residues.

95. The target recognition molecule as set forth in claim 94,
wherein further chemically linked to said electrostatically-charged segment is a base material immobilizing segment which is provided with a functional group for linkage to a base material.

96. The target recognition molecule as set forth in claim 95,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

97. The target recognition molecule as set forth in claim 1,
wherein said electrostatically-charged segment is a segment having a polydiallyldimethylammonium chloride building block represented by the following chemical formula (9) with n being not less than 3 nor more than 150.

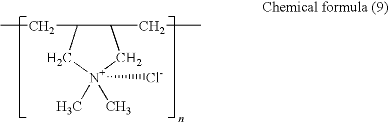

98. The target recognition molecule as set forth in claim 97,
wherein said target recognition peptide segment contains a cysteine residue, and wherein said connecting segment is chemically linked to elemental sulfur of said cysteine residue.

99. The target recognition molecule as set forth in claim 97,
wherein one end of said target recognition peptide segment is a cysteine residue, and wherein said connecting segment is chemically linked to elemental sulfur of said cysteine residue.

100. The target recognition molecule as set forth in claim 97,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

101. The target recognition molecule as set forth in claim 100,
wherein said target recognition peptide segment comprises a peptide which has not less than 3 nor more than 19 amino acid residues.

102. The target recognition molecule as set forth in claim 101,
wherein further chemically linked to said electrostatically-charged segment is a base material immobilizing segment which is provided with a functional group for linkage to a base material.

103. The target recognition molecule as set forth in claim 102,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

104. The target recognition molecule as set forth in claim 1,
wherein said electrostatically-charged segment is a segment having a polyvinylpyridine building block represented by the following chemical formula (10) with n being not less than 3 nor more than 150.

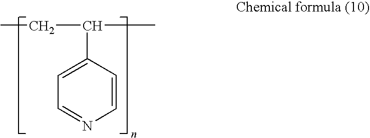

105. The target recognition molecule as set forth in claim 104,
wherein said target recognition peptide segment contains a cysteine residue, and wherein said connecting segment is chemically linked to elemental sulfur of said cysteine residue.

106. The target recognition molecule as set forth in claim 104,
wherein one end of said target recognition peptide segment is a cysteine residue, and wherein said connecting segment is chemically linked to elemental sulfur of said cysteine residue.

107. The target recognition molecule as set forth in claim 104,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

108. The target recognition molecule as set forth in claim 107,
wherein said target recognition peptide segment comprises a peptide which has not less than 3 nor more than 19 amino acid residues.

109. The target recognition molecule as set forth in claim 108,
wherein further chemically linked to said electrostatically-charged segment is a base material immobilizing segment which is provided with a functional group for linkage to a base material.

110. The target recognition molecule as set forth in claim 109,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

111. The target recognition molecule as set forth in claim 2,
wherein said target recognition peptide segment comprises a peptide which has not less than 3 nor more than 19 amino acid residues.

112. The target recognition molecule as set forth in claim 111,
wherein said target recognition peptide segment contains a cysteine residue, and wherein said connecting segment is chemically linked to elemental sulfur of said cysteine residue.

113. The target recognition molecule as set forth in claim 111,
wherein one end of said target recognition peptide segment is a cysteine residue, and wherein said connecting segment is chemically linked to elemental sulfur of said cysteine residue.

114. The target recognition molecule as set forth in claim 111,
wherein said connecting segment is chemically linked to either the N- or C-terminal of an amino acid residue of said target recognition peptide segment, and wherein said electrostatically-charged segment is chemically linked to a different site of said connecting segment from the aforementioned linkage site.

115. The target recognition molecule as set forth in claim 114,
wherein further chemically linked to said electrostatically-charged segment is a base material immobilizing segment which is provided with a functional group for linkage to a base material.

116. The target recognition molecule as set forth in claim 115,
wherein said connecting segment is chemically linked, at a linkable site of one end thereof, to said target recognition peptide segment, and is chemically linked, at a linkable site of the other end furthest away from said one end's linkable site, to said electrostatically-charged segment; and
wherein said electrostatically-charged segment is chemically linked, at a linkable site thereof furthest away from the site of its chemical linkage to said connecting segment, to said base material immobilizing segment.

117. The target recognition molecule as set forth in claim 116,
wherein said electrostatically-charged segment has no functional groups that become electrically charged to different polarities in the same solution.

118. The target recognition molecule as set forth in claim 117,
wherein said connecting segment has no functional groups that become electrically charged in the solution.

119. A target recognition molecule immobilization electrode base plate comprising:
a base plate which is provided with an electrode; and
a target recognition molecule as set forth in claim 1 immobilized onto said electrode of said base plate.

120. A specific molecule detection device comprising:
an electrode;
a detector for detecting a target molecule trapped by a target recognition molecule; and
a target recognition molecule as set forth in claim 1, immobilized onto said electrode.

121. A method for immobilizing a target recognition molecule as set forth in claim 3, onto an analytical chip provided with an electrode formed in a microchannel, said method comprising:
a step in which said target recognition molecule, having a target recognition peptide segment whose isoelectric point is 6 or less and an electrostatically-charged segment whose electrostatically-charged functional groups are those capable of becoming negatively electrically charged, is dissolved in a solution to thereby prepare a target recognition molecule containing solution with an adjusted solution pH value of 7 or greater; and
a step in which, with positive electric charges impressed to said electrode in said microchannel, said target recognition molecule containing solution is flowed in said microchannel so that said target recognition molecule is electrically trapped and retained on the surface of said electrode.

122. A method for immobilizing a target recognition molecule as set forth in claim 12, onto an analytical chip provided with an electrode formed in a microchannel, said method comprising:
a step in which said target recognition molecule, having a target recognition peptide segment whose isoelectric point is 8 or greater and an electrostatically-charged segment whose electrostatically-charged functional groups are those capable of becoming positively electrically charged, is dissolved in a solution to thereby prepare a target recognition molecule containing solution with an adjusted solution pH value of 7 or less; and
a step in which, with negative electric charges impressed to said electrode in said microchannel, said target recognition molecule containing solution is flowed in said microchannel so that said target recognition molecule is electrically trapped and retained on the surface of said electrode.

123. A method for immobilizing a target recognition molecule as set forth in claim 10, onto an analytical chip provided with an electrode formed in a microchannel, said method comprising:
a step in which said target recognition molecule, having a target recognition peptide segment whose isoelectric point is 6 or less and an electrostatically-charged segment whose electrostatically-charged functional groups are those capable of becoming negatively electrically charged, is dissolved in a solution to thereby prepare a target recognition molecule containing solution with an adjusted solution pH value of 7 or greater; and
a step in which, with positive electric charges impressed to said electrode in said microchannel, said target recognition molecule containing solution is flowed in said microchannel so that said target recognition molecule is electrically trapped on the surface of said electrode, thereby chemically linking a base material immobilizing segment of said target recognition molecule with said electrode.

124. A method for immobilizing a target recognition molecule as set forth in claim 19, onto an analytical chip provided with an electrode formed in a microchannel, said method comprising:
a step in which said target recognition molecule, having a target recognition peptide segment whose isoelectric point is 8 or greater and an electrostatically-charged segment whose electrostatically-charged functional groups are those capable of becoming positively electrically charged, is dissolved in a solution to thereby prepare a target recognition molecule containing solution with an adjusted solution pH value of 7 or less; and
a step in which, with negative electric charges impressed to said electrode in said microchannel, said target recognition molecule containing solution is flowed in said microchannel so that said target recognition molecule is electrically trapped on the surface of said electrode, thereby chemically linking a base material immobilizing segment of said target recognition molecule with said electrode.


SEQUENCE LISTING (17 sequences)

SEQ ID NO: 1 - protein, 7 aa, Homo sapiens
  MOD_RES (5)..(5): Phospho-Serine
LRRASLG

SEQ ID NO: 2 - protein, 8 aa, Artificial Sequence
  Description of Artificial Sequence Synthetic peptide
DDDDDDDD

SEQ ID NO: 3 - protein, 7 aa, Homo sapiens
  MOD_RES (1)..(1): N-term binding residue
GLSARRL

SEQ ID NO: 4 - protein, 8 aa, Artificial Sequence
  Description of Artificial Sequence Synthetic peptide
DDDDDDDD

SEQ ID NO: 5 - protein, 9 aa, Artificial Sequence
  Description of Artificial Sequence Synthetic peptide
DDDDDDDDC

SEQ ID NO: 6 - protein, 9 aa, Artificial Sequence
  Description of Artificial Sequence Synthetic peptide
DDDDDDDDC

SEQ ID NO: 7 - protein, 9 aa, Artificial Sequence
  Description of Artificial Sequence Synthetic peptide
DDDDDDDDC

SEQ ID NO: 8 - protein, 8 aa, Artificial Sequence
  Description of Artificial Sequence Synthetic peptide
LRRASLGC

SEQ ID NO: 9 - protein, 10 aa, Artificial Sequence
  Description of Artificial Sequence Synthetic peptide
RRRRRRRRRR

SEQ ID NO: 10 - protein, 8 aa, Artificial Sequence
  Description of Artificial Sequence Synthetic peptide
LRRASLGC

SEQ ID NO: 11 - protein, 11 aa, Artificial Sequence
  Description of Artificial Sequence Synthetic peptide
CRRRRRRRRRR

SEQ ID NO: 12 - protein, 12 aa, Artificial Sequence
  Description of Artificial Sequence Synthetic peptide
CRRRRRRRRRRC

SEQ ID NO: 13 - protein, 7 aa, Homo sapiens
  MOD_RES (7)..(7): C-term binding residue
LRRASLG

SEQ ID NO: 14 - protein, 8 aa, Artificial Sequence
  Description of Artificial Sequence Synthetic peptide
LRRASLGC

SEQ ID NO: 15 - protein, 7 aa, Homo sapiens
LRRASLG

SEQ ID NO: 16 - protein, 12 aa, Artificial Sequence
  Description of Artificial Sequence Synthetic peptide
CRRRRRRRRRRC

SEQ ID NO: 17 - protein, 7 aa, Homo sapiens
  MOD_RES (1)..(1): N-term binding residue
GLSARRL